SEQUENCE LISTING (51 sequences)

SEQ ID NO: 1 - DNA, 2699 bp, Homo sapiens
ggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaaaaaaaaaaaaaaaaaaaaaaaaaaaaaaa

SEQ ID NO: 2 - DNA, 3447 bp, Homo sapiens
ggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaaaaaaaaaa

SEQ ID NO: 3 - DNA, 5635 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 4 - DNA, 1830 bp, Homo sapiens
ggcacgagggtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgcaaaaaaaaaaaaaaaaaa

SEQ ID NO: 5 - DNA, 1667 bp, Homo sapiens
ggcacgaggcttccccttcccctcccagcgcgcccgcgcgccccgcggccctcggcgagcagctcggctccccccagcgctccccgggcccaaagatatggcaatggtagttagcagctggcgagatccgcaggacgacgtggccgggggcaaccccggcggccccaaccccgcagcgcaggcggcccgcggcggcggcggcggcgccggcgagcagcagcagcaggcgggctcgggcgcgccgcacacgccgcagaccccgggccagcccggagcgcccgccacccccggcacggcgggggacaagggccagggcccgcccggttcgggccagagccagcagcacatcgagtgcgtggtgtgcggggacaagtcgagcggcaagcactacggccaattcacctgcgagggctgcaaaagtttcttcaagaggagcgtccgcaggaacttaacttacacatgccgtgccaacaggaactgtcccatcgaccagcaccaccgcaaccagtgccaatactgccgcctcaagaagtgcctcaaagtgggcatgaggcgggaagcggttcagcgaggaagaatgcctccaacccagcccaatccaggccagtacgcactcaccaacggggaccccctcaacggccactgctacctgtccggctacatctcgctgctgctgcgcgccgagccctaccccacgtcgcgctacggcagccagtgcatgcagcccaacaacattatgggcatcgagaacatctgcgagctggccgcgcgcctgctcttcagcgccgtcgagtgggcccgcaacatccccttcttcccggatctgcagatcaccgaccaggtgtccctgctacgcctcacctggagcgagctgttcgtgctcaacgcggcccagtgctctatgccgctgcacgtggcgccgttgctggccgccgccggcctgcatgcctcgcccatgtctgccgaccgcgtcgtggccttcatggaccacatccgcatcttccaggagcaggtggagaagctcaaggcgctacacgtcgactcagccgagtacagctgcctcaaagccatcgtgctgttcacgtcagacgcctgtggcctgtcggatgcggcccacatcgagagcctgcaggagaagtcgcagtgcgcactggaggagtacgtgaggagccagtaccccaaccagcccagccgttttggcaaactgctgctgcgactgccctcgctgcgcaccgtgtcctcctccgtcatcgagcagctcttcttcgtccgtttggtaggtaaaacccccatcgaaactctcatccgcgatatgttactgtctgggagcagcttcaactggccttacatgtccatccagtgctcctagaccttgggcgcttcccacctgccccgtccccctagagactcagaggacccacctgggccaaggactccaaagccgcggggacaccgggaagtgcagcgggccaggcaggctgggtgggagggaggagggccgagacaggagcagcccacccagcagaaatacaatccgagctacaaagcatgggaaaaagagactcttttaggatcagatctgtgagcacgttggcgaggaaaaacaaaacaaacaaaaaaaagaaccttgtgtctgtctggtgaaaaaaaaaaaaaaaaaaaaaaaa

SEQ ID NO: 6 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 7 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 8 - protein, 626 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 9 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 10 - protein, 423 aa, Homo sapiens
MAMVVSSWRDPQDDVAGGNPGGPNPAAQAARGGGGGAGEQQQQAGSGAPHTPQTPGQPGAPATPGTAGDKGQGPPGSGQSQQHIECVVCGDKSSGKHYGQFTCEGCKSFFKRSVRRNLTYTCRANRNCPIDQHHRNQCQYCRLKKCLKVGMRREAVQRGRMPPTQPNPGQYALTNGDPLNGHCYLSGYISLLLRAEPYPTSRYGSQCMQPNNIMGIENICELAARLLFSAVEWARNIPFFPDLQITDQVSLLRLTWSELFVLNAAQCSMPLHVAPLLAAAGLHASPMSADRVVAFMDHIRIFQEQVEKLKALHVDSAEYSCLKAIVLFTSDACGLSDAAHIESLQEKSQCALEEYVRSQYPNQPSRFGKLLLRLPSLRTVSSSVIEQLFFVRLVGKTPIETLIRDMLLSGSSFNWPYMSIQCS

SEQ ID NO: 11 - DNA, 2549 bp, Homo sapiens
ggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcgggagccggccggagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaaaaaaaaaaaaaaaaaaaaaaaaaaaaaaa

SEQ ID NO: 12 - protein, 598 aa, Homo sapiens; identical to SEQ ID NO: 6

SEQ ID NO: 13 - DNA, 1842 bp, Homo sapiens
ggaggcagcaccacatccagagctcgctcagctgctgcccagcctcggctgtgaggataggctggctgggcagcacgtctctccccacagggctccctgagaccaccaggaagagcccccaaccaatcttgggattctcccttcgtgcggttgtctgggacctttttccagggtcaaagcagatcgtgaggaggaagctgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaaggtaccgcgcagccccaggtggggccttttgttggaaatggagagaggctggcctcatcccattgggacctgtggtctccccctgggttctcctcctagctaagtcctgtcctgcagggtgggatcagccctgccaggtgggccgccttcctggagacccgtagatgccagggctggaagctttcatttgccgggacactcgggcccatgggattgcacagagctggagggaggggtgagataggggcagataggagctgcaggggtgcctggcgagcctctggttttcctctgctcctctgcctgtcctctcccaactcaaggttctagtgggaaggggtgcccccaggctctcatgttcctggcgtgagatgaaaggatccctgcggagggtttggttcttgagggctgggggtggacttgggaacaggctgtgtgtttgtcccagcgatggtgcctgcttagcttcccgtccccaccccccagccccttggccctctcctgtctgccctagggagaaggcaggtggacaagggcccatgaaaaaatacaggtgtctagactgccagggagaccctggcccccagtagtgtgtcctggggacttcctcagagcgagaaacctcccccaatgtcttcaagacttttctctccccccgcccaaccccgtctctccctcccttgccacccaaatgttagaaaaatagctgtgaacagagagcgcttttgtctgcaatggcagcaggatctggacggtcccctcccc

SEQ ID NO: 14 - protein, 325 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKVPRSPRWGLLLEMERGWPHPIGTCGLPLGSPPS

SEQ ID NO: 15 - RNA, 19 nt, Artificial
  sense strand siRNA against NR4A1
guucgaggacuuccaggug

SEQ ID NO: 16 - RNA, 19 nt, Artificial
  antisense strand siRNA against NR4A1
caccuggaaguccucgaac

SEQ ID NO: 17 - RNA, 19 nt, Artificial
  sense strand siRNA against NR4A1
ggaaguuguccgaacagac

SEQ ID NO: 18 - RNA, 19 nt, Artificial
  antisense strand siRNA against NR4A1
gucuguucggacaacuucc

SEQ ID NO: 19 - RNA, 19 nt, Artificial
  sense strand siRNA against NR4A1
gaucuucauggacacgcug

SEQ ID NO: 20 - RNA, 19 nt, Artificial
  antisense strand siRNA against NR4A1
cagcguguccaugaagauc

SEQ ID NO: 21 - DNA, 8 bp, Homo sapiens
aaaggtca

SEQ ID NO: 22 - DNA, 8 bp, Homo sapiens
aaatryca

SEQ ID NO: 23 - RNA, 19 nt, Artificial
  sense strand siRNA against NR4A2
cuacagcacaggcuacgac

SEQ ID NO: 24 - RNA, 19 nt, Artificial
  antisense strand siRNA against NR4A2
gucguagccugugcuguag

SEQ ID NO: 25 - RNA, 19 nt, Artificial
  sense strand siRNA gainst NR4A2
ucgcugucaguacugccga

SEQ ID NO: 26 - RNA, 19 nt, Artificial
  antisense strand siRNA against NR4A2
ucggcaguacugacagcga

SEQ ID NO: 27 - RNA, 19 nt, Artificial
  sense strand siRNA against NR4A2
gagaguggaagaacugcaa

SEQ ID NO: 28 - RNA, 19 nt, Artificial
  antisense strand siRNA against NR4A2
uugcaguucuuccacucuc

SEQ ID NO: 29 - DNA, 6382 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagatagtgtgtatatatatatatatgggtgggtgttttgttgcagctgctgatctttttctttgcagatggtacaaactctcccgagtcaatttcctgggcctatgtccccacctagctgactgaagttatcaacaggggtccagtttgtgcaggctgctagccctattggaagagtggggatgaggtgggagaaagcaaccacaacgtgtgtgggcaacctcaattggcactcataaaatgttagaatgtcaactctctcccttggccactaaatctctcacagggtagtttttcttgcctaactcaggtttacaaatcaatgtgtatgccttgggggaccaatggcctctttcctcccaaataaaccactggctttctctttgtccccctaggttatagctgaggagcccactccaattagtttataggattcaaagcctctttttaaaaacatctctgagcttatgaggaaagacttcaagtttcccaaatctagtggaggacagggcaagggaggaaagataggtacaggagtccacaggaggccaggttttggcacccctttgtcaggaattcagcttccttactagggatgaagaaaataagtgtggggctttgtgtctatgctaccagaaggaggagaggatgacacttcctctctgtttcccagattagagaacagtgaacccaatgctgcctgttggctagaaaacaagtgttaacttgcttctgagagacccttttctctgtccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 30 - protein, 626 aa, Homo sapiens; identical to SEQ ID NO: 8

SEQ ID NO: 31 - DNA, 4983 bp, Homo sapiens
caggccctcatcaccttttttcaagtcaagatttcatcccatacatgcatgactcaatcagatttggaaatgtggatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 32 - protein, 637 aa, Homo sapiens
MHDSIRFGNVDMPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 33 - DNA, 2588 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgctcttgtccgagctttaacagactcaacacccagagatcttgattattccagagtaagttttatgatttcctgctttcaaatgaatgatcagggtctctatttatggctactagtaataagagttgattgaatgattttgtgtctggcaccatgttagacagttttcatactttttctatatttctcgcttcatttagcaattcagtgcatccattgcagcaaataatttttgccttattgaatctctaaatgccttaacaagtgaccctgacagtgctgcacctgtcatacacattgttgcaggattcctggtggttgtgccaatgaaaatctgcacagacaaactacaatttgtagatttatctcgtgatctagacaaagtgactactgttttttttcatattgtgttcaaaccatctgggtgagcctcaagttattactaagcagtttatccaattgcatcagcattgattgacctgctgcttattcagagggattagaccattggtggagagaagcgagctctggctgtgacatcagttgtcaggagaactgggatggaacgccacctctgctacttcctgcctgtgtcacctggttcctgcttttggctctctcctgcctacagtggaaatggctacaatatttaacctcaga

SEQ ID NO: 34 - protein, 443 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRVSFMISCFQMNDQGLYLWLLVIRVD

SEQ ID NO: 35 - RNA, 21 nt, Artificial
  sense strand siRNA against NR4A3
guguucugcaauggacuuguc

SEQ ID NO: 36 - RNA, 21 nt, Artificial
  sense strand siRNA against NR4A3
gacaagcucuuccuggacacc

SEQ ID NO: 37 - RNA, 21 nt, Artificial
  sense strand siRNA against NR4A3
guucaccuucccugucgaucc

SEQ ID NO: 38 - RNA, 21 nt, Artificial
  sense strand siRNA against NR2F6
aacuugcggccaaacuuuccc

SEQ ID NO: 39 - RNA, 19 nt, Artificial
  sense strand siRNA against NR2F6
aacuuuuugggggugcagc

SEQ ID NO: 40 - RNA, 21 nt, Artificial
  sense strand siRNA against NR2F6
aagcauuacggugucuucacc

SEQ ID NO: 41 - RNA, 21 nt, Artificial
  sense strand siRNA against NR2F6
aaccgugacugccagaucgac

SEQ ID NO: 42 - RNA, 19 nt, Artificial
  sense strand siRNA against NR2F6
aagacgcccauugagacug

SEQ ID NO: 43 - RNA, 52 nt, Artificial
  ribozyme targeting NR2F6
caggugaaagaaccguaccagagaaacacacguugugguauauuaccuggua

SEQ ID NO: 44 - DNA, 52 bp, Artificial
  ribozyme targeting NR2F6
cttcttgaagaagcagaccagagaaacacacguugugguauauuaccuggua

SEQ ID NO: 45 - DNA, 52 bp, Artificial
  ribozyme targeting NR2F6
atgtgtctagaataggaccagagaaacacacguugugguauauuaccuggua

SEQ ID NO: 46 - RNA, 19 nt, Artificial
  sense strand siRNA against NR2F1
uacugccgccucaagaagu

SEQ ID NO: 47 - RNA, 19 nt, Artificial
  antisense strand siRNA against NR2F1
acuucuugaggcggcagua

SEQ ID NO: 48 - RNA, 19 nt, Artificial
  sense strand siRNA against NR2F1
ggcgcuacacgucgacuca

SEQ ID NO: 49 - RNA, 19 nt, Artificial
  antisense strand siRNA against NR2F1
ugagucgacguguagcgcc

SEQ ID NO: 50 - RNA, 19 nt, Artificial
  sense strand siRNA against NR2F1
cuggccuuacauguccauc

SEQ ID NO: 51 - RNA, 19 nt, Artificial
  antisense strand siRNA against NR2F1
gauggacauguaaggccag

(19) United States
(12) Patent Application Publication
(10) Pub. No.: US 2007/0184459 A1
(43) Pub. Date: Aug. 9, 2007

(54) Methods of inhibiting cancer growth by binding to nuclear receptors

(75) Inventors: Henry Li, Carlsbad, CA (US); Ning Ke, San Diego, CA (US); Mirta Grifman, San Diego, CA (US); Gisela Claasen, San Diego, CA (US); Xiuyuan Hu, San Diego, CA (US); Kristen Defife, San Diego, CA (US); Cellia Habita, La Jolla, CA (US); Wufang Fan, San Diego, CA (US); Kristina Rhoades, Crowley, TX (US); Philip Tan, San Diego, CA (US); Flossie Wong-Staal, San Diego, CA (US)

Correspondence Address: David Spolter;LAW OFFICE OF DAVID SPOLTER, c/o G. Singer, 30 South Adelaide Ave., #5M, Highland Park, NJ, 08904, US

(73) Assignee: Immusol Incorporated

(21) Appl. No.: 11/351,522

(22) Filed: Feb. 3, 2006

(51) Int. Cl. C12Q 1/68; G01N 33/574; C12N 15/09

(52) U.S. Cl. 435/6; 435/7.23; 435/455

(57) ABSTRACT

The present invention provides methods for identifying agents useful for inhibiting cancer cells by binding to various nuclear receptor proteins, or to the genes or RNA encoding such proteins.

### FIELD OF THE INVENTION

The present invention is directed to methods of identifying agents useful for the treatment of cancer that bind to nuclear receptor proteins, or to the genes or mRNA encoding such proteins. The invention is also directed to methods for the treatment of cancer by administering agents that bind to nuclear receptor proteins, or to the genes or mRNA encoding such proteins.

### BACKGROUND OF THE INVENTION

Cancer is a major cause of mortality worldwide. Despite advancements in diagnosis and treatment, there remains a great need for novel methods of treating cancer and for identifying novel agents that inhibit the growth of cancer cells. The present invention satisfies this need and provides additional benefits as well.

### SUMMARY OF THE INVENTION

The present invention centers on the discovery that knockdown of a number of nuclear receptor proteins expressed in cancerous tissue, as well as the genes or mRNA encoding such proteins, results in the death of the cancer cells, reduction in their size, a decrease in their growth or an increase in sensitization to undergo apoptosis. Accordingly, the present invention provides methods for identifying agents useful in the treatment of cancer, such methods based upon the binding of such agents to the nuclear receptor proteins of the invention, or to the genes or mRNA encoding such proteins. Such methods include introducing an agent into cancerous cells in which one or more of the nuclear receptors of the invention is expressed, and determining the effect of such agent on the cells. An agent that effectively binds to one of the nuclear receptors of the invention and thereby causes a decrease in cancer cell proliferation, or an increase in cell death (apoptosis), or otherwise decreases cancerous growth, will be useful for the treatment of cancer. Representative agents include antisense oligonucleotides, ribozymes, siRNAs, monoclonal and polyclonal antibodies, and small organic molecules.

### BRIEF DESCRIPTION OF THE DRAWINGS

FIG. 1 shows the DNA sequence of the nuclear receptor NR4A1 (SEQ ID NO:1).

FIG. 2 shows the DNA sequence of the nuclear receptor NR4A2 (SEQ ID NO:2).

FIG. 3 shows the DNA sequence of the nuclear receptor NR4A3 (SEQ ID NO:3).

FIG. 4 shows the DNA sequence of the nuclear receptor NR2F6 (SEQ ID NO:4).

FIG. 5 shows the DNA sequence of the nuclear receptor NR2F1 (SEQ ID NO:5).

FIG. 6 shows the amino acid sequence of the protein encoded by NR4A1 (SEQ ID NO:6).

FIG. 7 shows the amino acid sequence of the protein encoded by NR4A2 (SEQ ID NO:7).

FIG. 8 shows the amino acid sequence of the protein encoded by NR4A3 (SEQ ID NO:8).

FIG. 9 shows the amino acid sequence of the protein encoded by NR2F6 (SEQ ID NO:9).

FIG. 10 shows the amino acid sequence of the protein encoded by NR2F1 (SEQ ID NO:10).

FIG. 11 shows the results of real-time PCR (RT-PCR) experiments performed to assess the expression levels of NR4A1 (SEQ ID NO:1) in several cancer cell lines.

FIG. 12 shows that NR4A1 mRNA knockdown in HeLa cells results in growth inhibition as measured both by anchorage-dependent growth (FIG. 12A) and anchorage-independent growth (FIG. 12B). FIG. 12C shows NR4A1 mRNA knockdown by siRNAs in HeLa cells.

FIG. 13 shows the effect of NR4A1 mRNA knockdown on anchorage-independent cell growth or survival in the following cell lines: in DLD-1 (FIG. 13A), AsPC1 (FIG. 13B), U87 (FIG. 13C), A2058 (FIG. 13D) and PC3 (FIG. 13E).

FIGS. 14A, 14B and 14C show the results of caspase-based apoptosis assays in three separate cell lines that indicate that NR4A1 mRNA knockdown by siRNAs causes an increase in caspase activity (i.e. an increase in apoptotic cell death). FIG. 14D shows the result of an ELISA assay on HeLa cells transfected with siRNA targeting NR4A1.

FIG. 14E compares NR4A1 knockdown by siRNA in a prostrate cancer cell line (PC-3M/N) and a non-cancerous prostate cell line (PrEC).

FIG. 15 shows the effect in three separate cell lines of transient transduction of siRNA against NR4A1.

FIG. 16 shows the results of real-time PCR (RT-PCR) experiments performed to assess the expression levels of NR4A2 (SEQ ID NO:2) in several cancer cell lines.

FIG. 17 shows the effect of siRNA-mediated NR4A2 mRNA knockdown on Hela cell growth, as tested in anchorage-dependent growth (FIG. 17A) and anchorage-independent growth (FIG. 17B). FIG. 17C shows real-time PCR analysis of NR4A2 mRNA in HeLa cells after knockdown by siRNAs.

FIG. 18 shows the effect of NR4A2 knockdown (as measured by anchorage independent growth) in the following cell lines: DLD-1 (FIG. 18A), AsPC1 (FIG. 18B), U87 (FIG. 18C) and A2058 (FIG. 18D).

FIG. 19A shows the effect on cell growth in four separate cell lines after stable transduction of siRNA against NR4A2. FIG. 19B shows NR4A2 mRNA expression levels in these same four cell lines. FIG. 19C shows the role of NR4A2 in anoikis in HeLa and HCT116 cells after stable transduction with siRNA against NR4A2.

FIG. 20 shows the results of real-time PCR (RT-PCR) experiments performed to assess the expression levels of NR4A3 (SEQ ID NO:3) in several cancer cell lines.

FIG. 21A shows soft-agar growth of HeLa cells transfected with siRNAs against NR4A3. FIG. 21B shows real time RT-PCR measurement of NR4A3 message in HeLa cells treated with siRNAs against NR4A3.

FIG. 22 shows percentage survival (WST-1 assay after addition of C₂ ceramide) of A172 cells after transfection with five siRNAs targeting NR2F6 (SEQ ID NO:4).

FIG. 23 shows increase in apoptosis in JH-MG cells after transfection with four siRNAs targeting NR2F6 and addition of Fas.

FIG. 24 shows increase in apoptosis in AsPC1 cells after transfection with two siRNAs targeting NR2F6 and addition of Fas.

FIG. 25 shows the effects of siRNA against NR2F1 on A2058 cell sensitization to apoptosis induced by growth factor deprivation (FIG. 25A) and by Brefeldin A (FIG. 25B). FIG. 25C shows the down regulation by siRNAs of NR2F1 mRNA in A2058 cells.

FIG. 26 shows the effects on cell soft agar growth by siRNAs against NR2F1 (SEQ ID NO:5) in HeLa (FIG. 26A), DLD-1 (FIG. 26B) and U87 (FIG. 26C) cells.

FIG. 27 shows the DNA sequence of variant 2 of NR4A1 (SEQ ID NO:11); and

FIG. 28 shows the encoded amino acid sequence (SEQ ID NO:12).

FIG. 29 shows the DNA sequence of variant 3 of NR4A1 (SEQ ID NO:13); and

FIG. 30 shows the encoded amino acid sequence (SEQ ID NO:14).

FIG. 31 shows the DNA sequence of variant 2 of NR4A3 (SEQ ID NO:29); and

FIG. 32 shows the encoded amino acid sequence (SEQ ID NO:30).

FIG. 33 shows the DNA sequence of variant 3 of NR4A3 (isoform b; SEQ ID NO:31); and

FIG. 34 shows the encoded amino acid sequence (SEQ ID NO:32).

FIG. 35 shows the DNA sequence of variant 4 of NR4A3 (isoform c; SEQ ID NO:33); and

FIG. 36 shows the encoded amino acid sequence (SEQ ID NO:34).

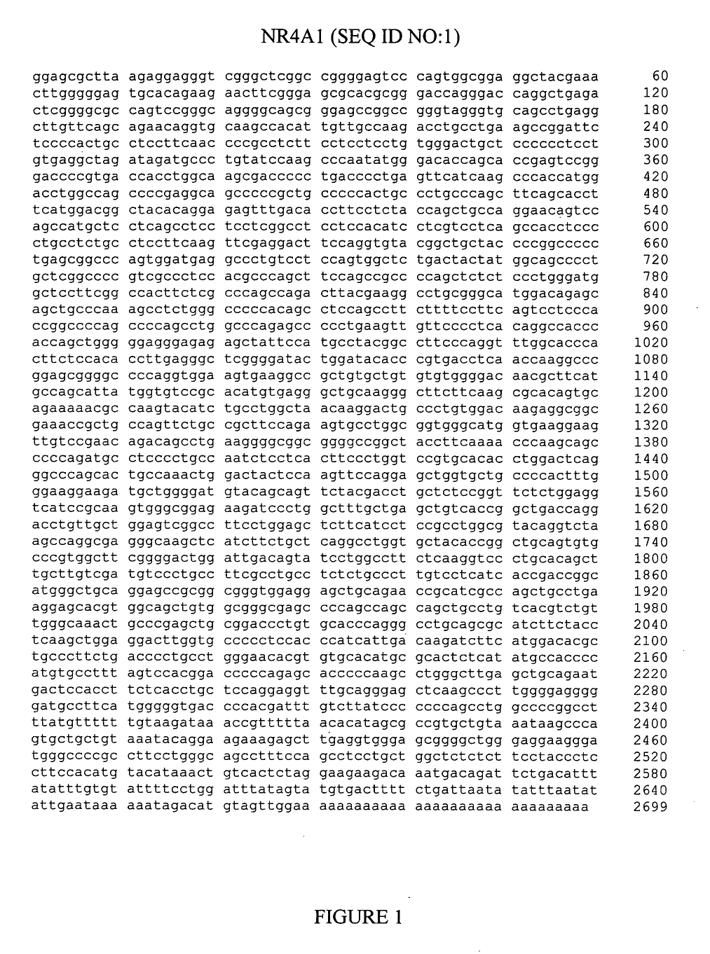

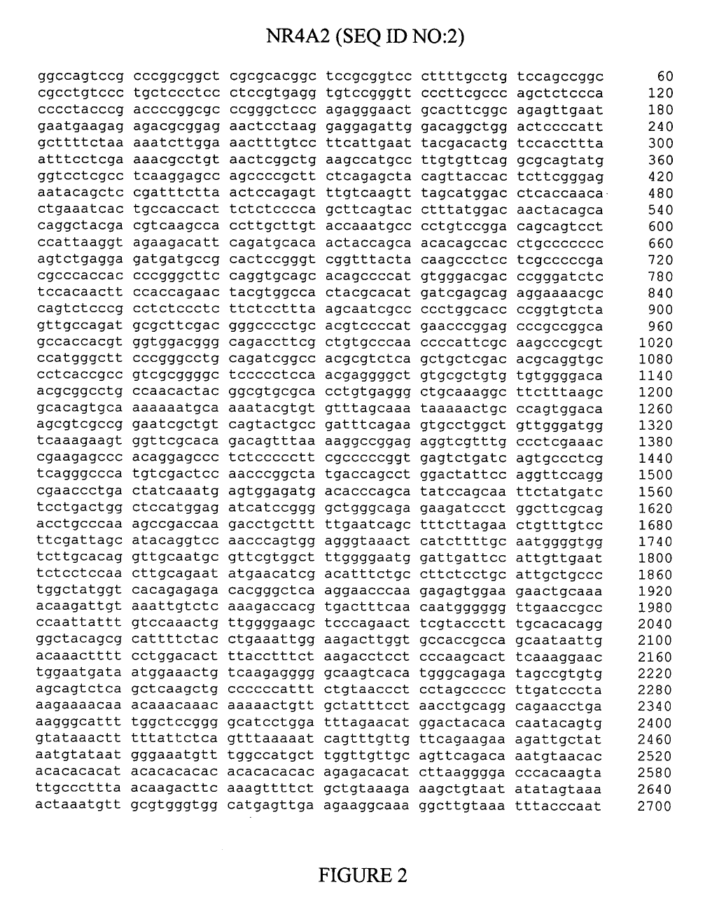

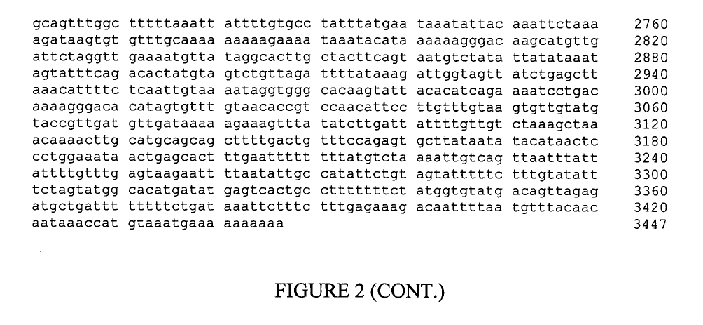

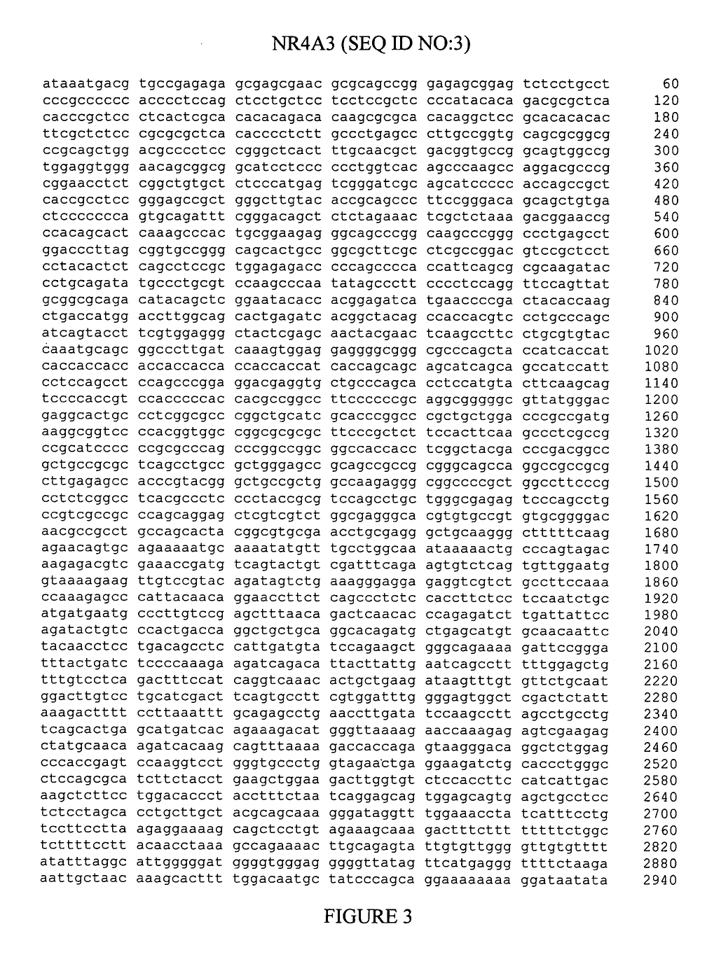

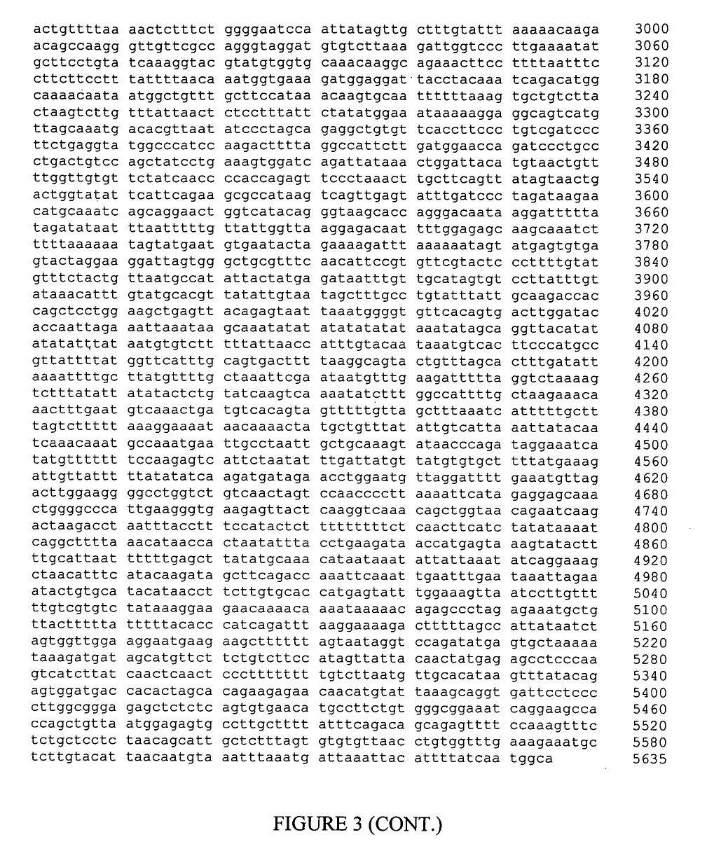

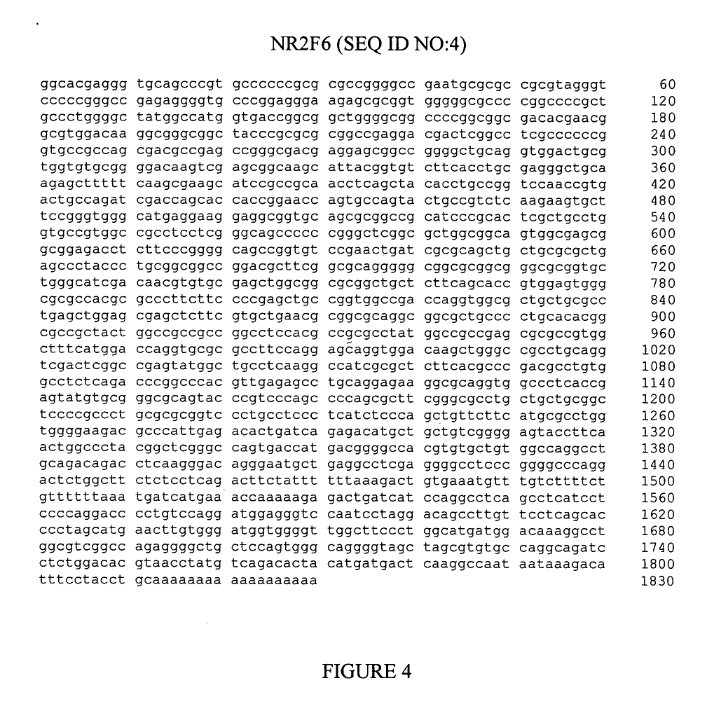

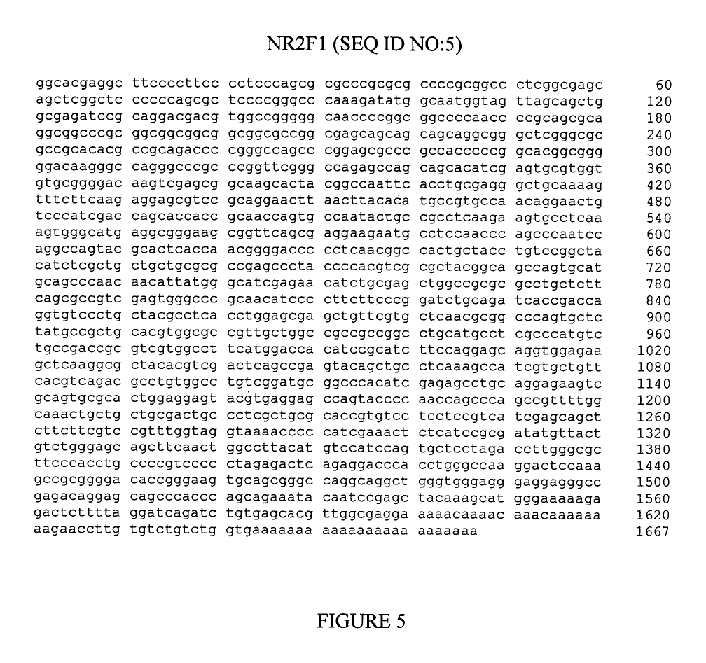

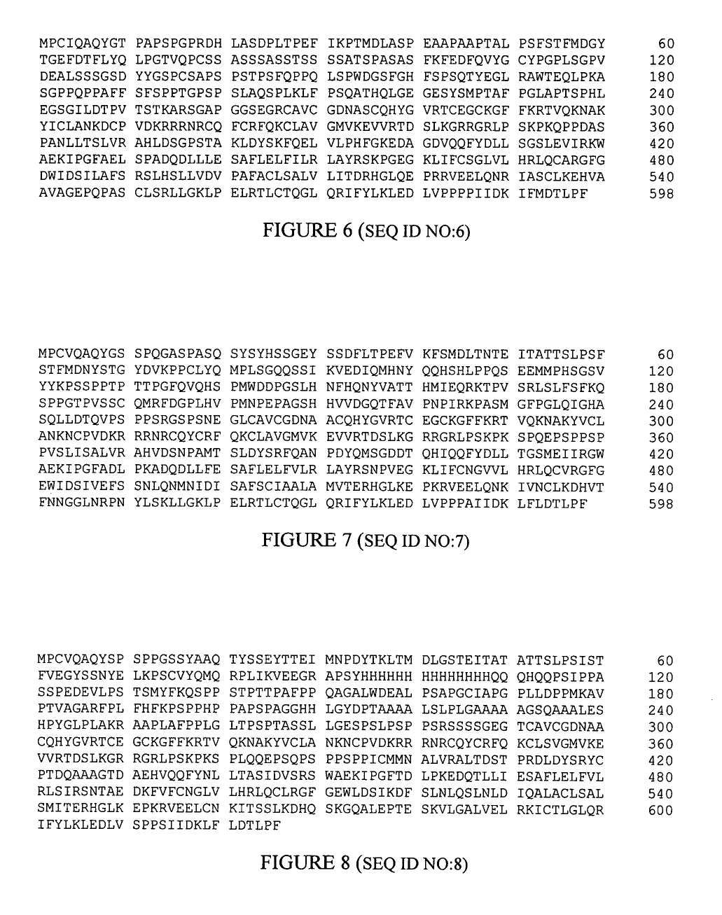

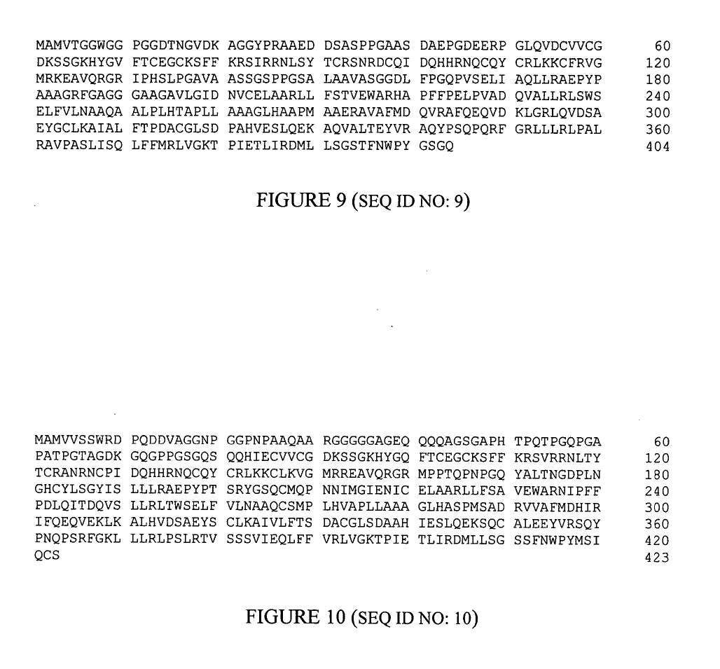

### DETAILED DESCRIPTION OF THE INVENTION

The present invention centers on the discovery of the correlation between the knockdown of various nuclear receptor proteins, as well as the genes and mRNA encoding such proteins, and the treatment of cancer.

Specifically, the invention provides a method of identifying a molecule useful for the treatment of cancer by introducing the molecule into cancer cells, where it binds to a protein encoded by one of the nuclear receptors, NR4A1, NR4A2, NR4A3, NR2F6 or NR2F1 (SEQ ID NOS:1, 2, 3, 4 or 5, respectively) or to the mRNA of SEQ ID NOS:1, 2, 3, 4 or 5. The method further includes measuring the level of “inhibition” of the cancerous cells. “Inhibition” means any one or more of the following: a) a decrease in cancer cell division (proliferation); b) an increase in the sensitivity of the cells to undergo apoptosis; or c) an increase in cell death (apoptosis).

Examples of cancer cells useful for the practice of the invention include HeLa cells, A2058 cells, DLD1 cells, T47D cells, ASPC1 cells and JH-MG cells.

The invention further provides a method of identifying a molecule that inhibits cancer cells by introducing the molecule into cells, where the molecule binds to a domain of a nuclear receptor protein, specifically a domain of SEQ ID NOS:6, 7, 8, 9 or 10. These nuclear receptor proteins each have a zinc finger binding domain (ZF) and a ligand binding domain (HOLI). In particular, these domains are: amino acid 264 to amino acid 335 of SEQ ID NO:6 (ZF), amino acid 408 to amino acid 566 of SEQ ID NO:6 (HOLI), amino acid 260 to amino acid 331 of SEQ ID NO:7 (ZF), amino acid 408 to amino acid 566 of SEQ ID NO:7 (HOLI), amino acid 289 to amino acid 360 of SEQ ID NO:8 (ZF), amino acid 439 to amino acid 594 of SEQ ID NO:8 (HOLI), amino acid 53 to amino acid 124 of SEQ ID NO:9 (ZF), amino acid 204 to amino acid 364 of SEQ ID NO:9 (HOLI), amino acid 83 to amino acid 154 of SEQ ID NO:10 (ZF) and amino acid 221 to amino acid 381 of SEQ ID NO:10 (HOLI). The method of the invention further provides measuring the level of inhibition of the cells.

Measurement of cancer cell inhibition can be done, for example, by means of an apoptosis assay, where an increase in the level of apoptosis indicates that the molecule introduced into the cell system inhibits the cancer cells. Measurement of inhibition can also be made by means of an assay that measures cell proliferation, where a decrease in the rate of cell division indicates that the molecule inhibits the cancer cells.

The invention further provides a method of inhibiting cancer cells by introducing into the cells a molecule that binds to a compound comprising SEQ ID NOS:1, 2, 3, 4 or 5, or comprising the RNA correlate of SEQ ID NOS:1, 2, 3, 4 or 5.

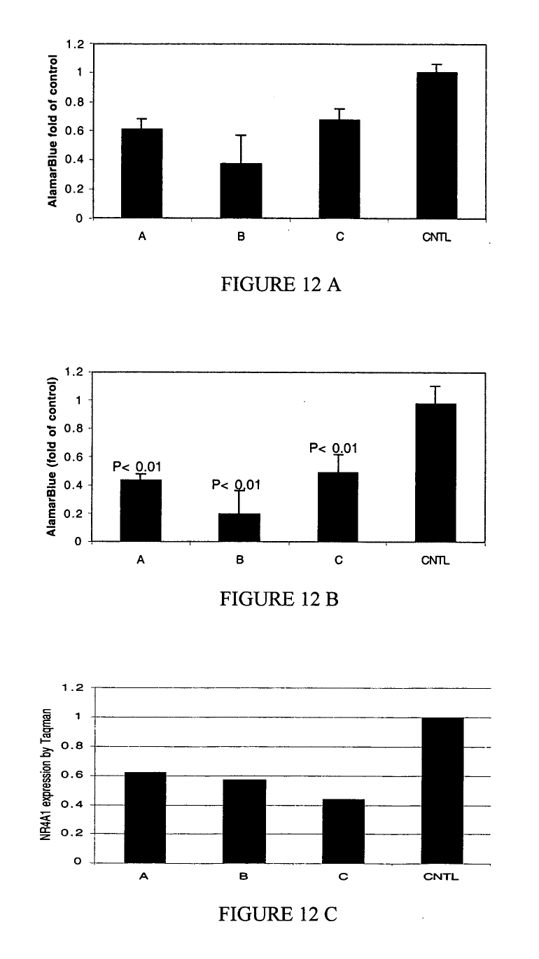

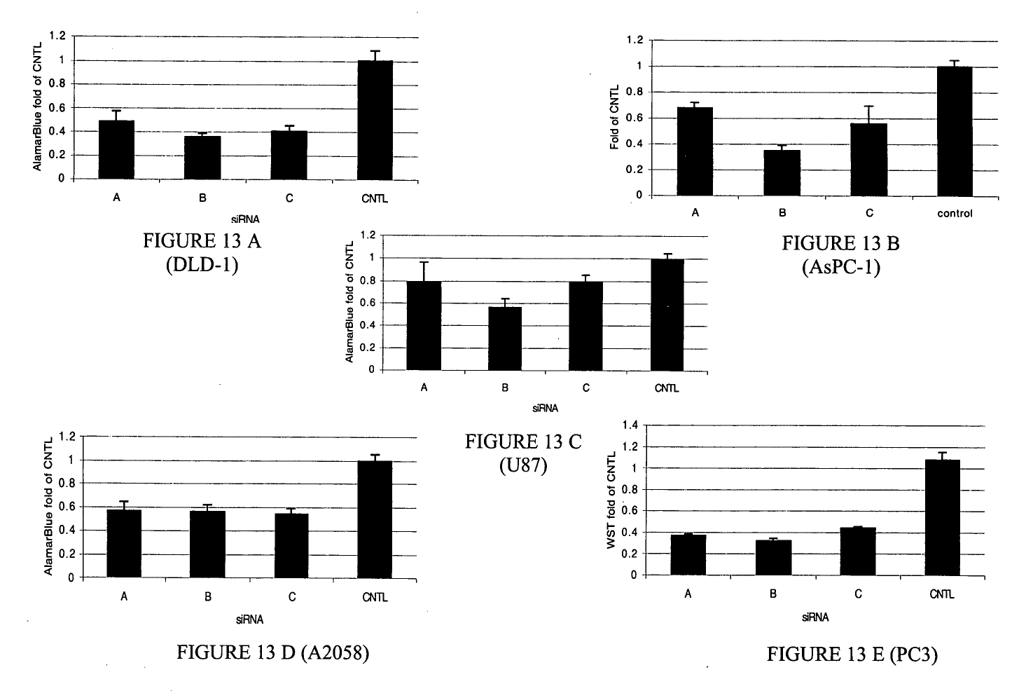

An example of such a molecule is an siRNA. Introduction into HeLa cells of three separate siRNA molecules targeting the nuclear receptor NR4A1 (SEQ ID NO:1; see Example 1) each caused reduction of the mRNA of NR4A1 and reduction of both anchorage dependent growth and soft-agar growth of the HeLa cells (see FIGS. 12A and 12B). Cell growth was also reduced in three other cancer cell lines (DLD, AsPC1 and U87) using siRNA that targeted the mRNA of NR4A1. See FIG. 13.

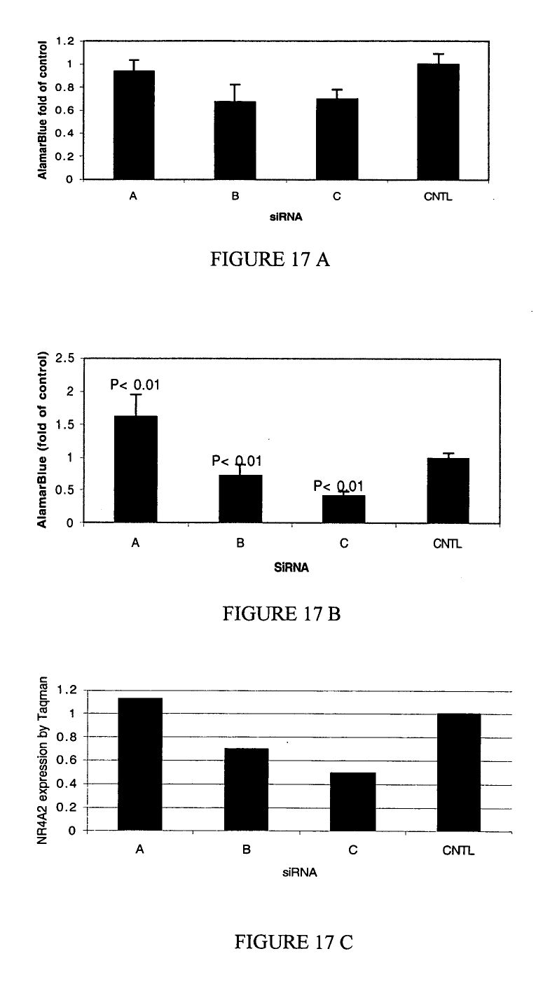

Moreover, the introduction into HeLa cells of two separate siRNA molecules targeting the nuclear receptor NR4A2 (SEQ ID NO:2) each caused reduction of the mRNA of NR4A2 (see Example 2) and reduction in anchorage dependent growth and soft-agar growth of the HeLa cells (see FIGS. 17A and 17B).

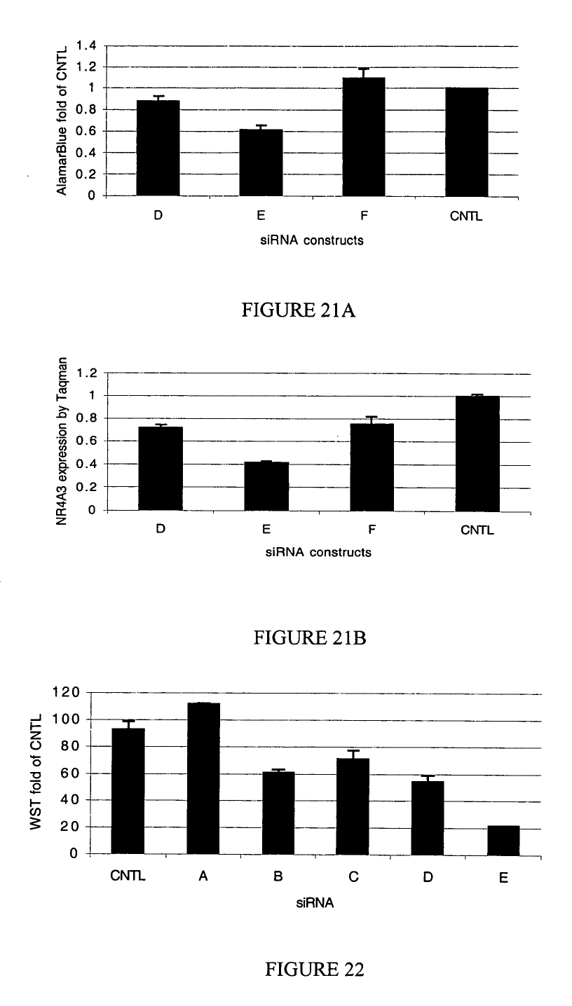

Furthermore, another siRNA of the invention (labeled “E” in FIG. 21A) against the nuclear receptor of the invention NR4A3 (SEQ ID NO:3) caused significant reduction in soft-agar growth of HeLA cells. In addition, this siRNA construct, as well as two others (labeled “D” and “F” in FIGS. 21A and 21B) against the nuclear receptor of the invention NR4A3 (SEQ ID NO:3) reduced the level of mRNA in HeLA cells (see FIG. 21B).

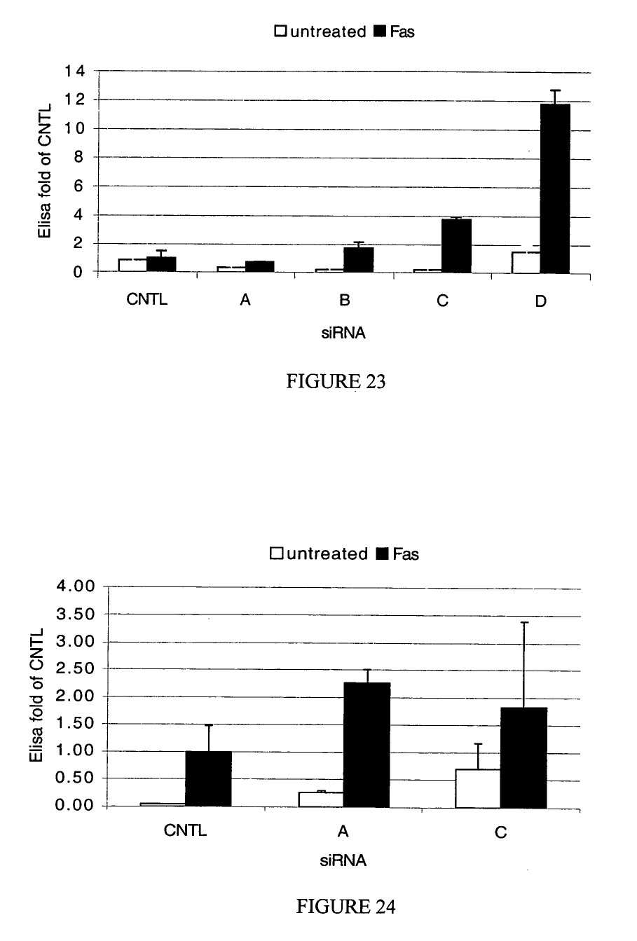

In addition, five separate siRNA constructs targeting the nuclear receptor NR2F6 (SEQ ID NO:4; see Example 4) reduced the level of mRNA of NR2F6, and increased the level of sensitivity to apoptosis in the glioblastoma cell line JH-MG cells (see FIG. 23) and pancreatic cell line AsPC1 cells (see FIG. 24).

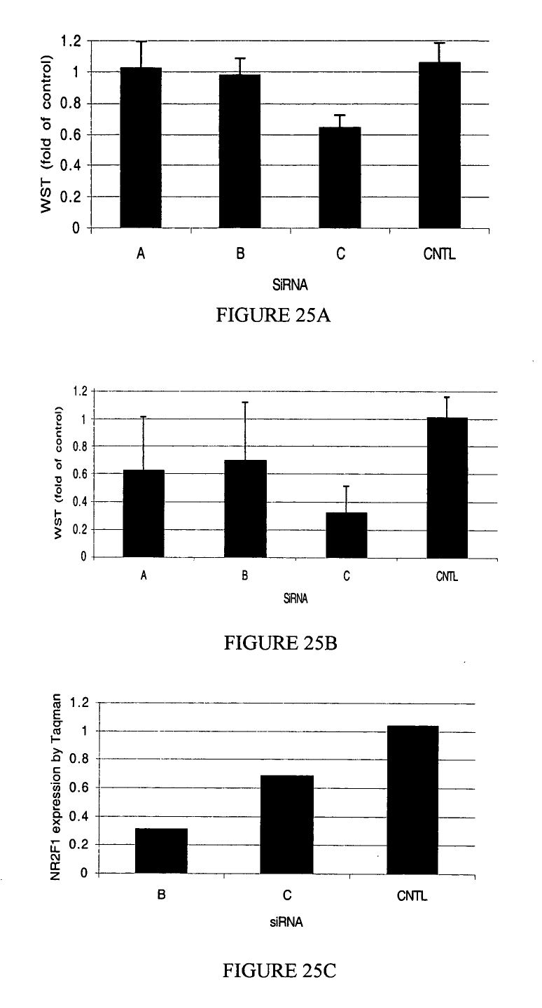

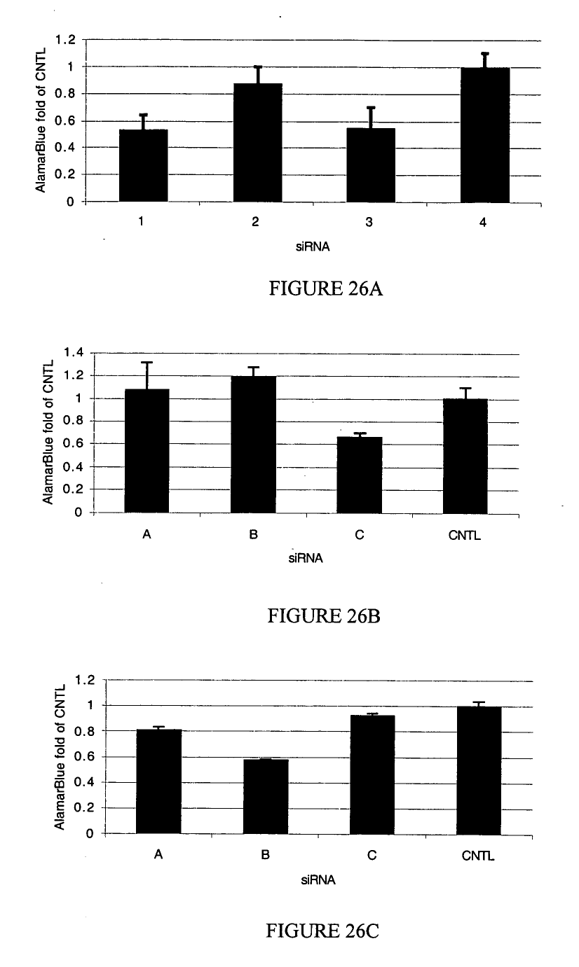

Moreover, when three separate siRNA constructs against the mRNA of the nuclear receptor NR2F1 (SEQ ID NO:5) were introduced to the melanoma cell line A2058, the sensitivity of the cells to undergo apoptosis was measurably increased (see FIG. 25). These siRNAs also reduced soft-agar growth of the cervical cancer cell line HeLa and of the glioma cell line U87 (see FIG. 26).

In addition to siRNA constructs, a molecule of the invention also includes ribozymes, antisense molecules, antagonizing antibodies and small organic molecules that target or bind to one of the nuclear receptors of the invention. Thus, three ribozymes (SEQ ID NOS:43, 44 and 45) directed at NR2F6 (SEQ ID NO:4) conferred the same effect as the siRNAs targeting NR2F6, as described above. See Example 5, Section 7.

An example of an apoptosis assay is the Annexin-V binding assay. This assay is based on the relocation of phosphatidylserine to the outer cell membrane. Viable cells maintain an asymmetric distribution of different phospholipids between the inner and outer leaflets of the plasma membrane. Choline-containing phospholipids such as phosphatidylcholine and sphingomyelin are primarily located on the outer leaflet of viable cells and aminophospholipids such as phosphatidylethanolamine and phosphatidylserine (PS) are found at the cytoplasmic (inner) face of viable cells. The distribution of phospholipids in the plasma membrane changes during apoptosis. In particular, PS relocates from the cytoplasmic face to the outer leaflet so called PS exposure. The extent of PS exposure can distinguish apoptotic cells from the non-apoptotic cells.

Annexin-V is a 35-36 kDa calcium-dependent phospholipid binding protein with high affinity for PS (kDa˜5×10-10 M). When labeled with a fluorescent dye, Annexin-V can be used as a sensitive probe for PS exposure on the outer leaflet of the cell membrane. The binding of Annexin-V conjugates such as Annexin-V FITC to cells permits differentiation of apoptotic cells (Annexin-V positive) from non-apoptotic cells (Annexin-V negative). Annexin-V binding is observed under two conditions. The first condition is observed in cells midway through the apoptosis pathway. Phosphatidylserine translocates to the outer leaflet of the cell membrane. The second condition is observed in very late apoptosis or when the cells become necrotic and membrane permeabilization occurs. This membrane permeabilization allows Annexin-V to enter cells and bind to phosphatidylserine on the cytoplasmic face of the membrane. Since other causes besides apoptosis can result in necrosis, it is important to distinguish between necrotic and apoptotic cells. Membrane permeabilization also permits entry of other materials to the interior of the cell, including the fluorescent DNA-binding dye propidium iodide. Utilizing dual staining methodology, apoptotic populations can be distinguished from necrotic populations. For example, using the Annexin V-propidium iodide (PI) double staining regime, three populations of cells are distinguishable in two color flow cytometry. See Boersma, et al., Cytometry, 24:123-130 (1996); Martin, et al., J. Exp. Med., 182:1545-1556 (1995).

Another example of an apoptosis assay is the caspase 3/7 assay. Briefly, caspases are synthesized as inactive pro-enzymes or pro-caspases. In apoptosis, the pro-caspases are processed by proteolytic cleavage to form active enzymes. For example, caspase-3 exists in cells as an inactive 32 kDa proenzyme, called pro-caspase-3. Pro-caspase-3 is cleaved into active 17 and 12 kDa subunits by upstream proteases to become active caspase-3. Caspases-2, -8, -9 and -10 are classified as signaling or “upstream” in the apoptosis pathway because long prodomains allow association with cell surface receptors such as FAS (CD95), TNFR-1 (CD120a), DR-3 or CARD domains. This observation suggests a proteolytic cascade as a mechanism for signaling. A proteolytic cascade exists that would activate the terminal event required for apoptosis in a way similar to that of the coagulation cascade seen with the closely related family of serine proteases. For example, caspase-4 activates pro-caspase-1; caspase-9 activates pro-caspase-3; and caspase-3 cleaves pro-caspase-6 and pro-caspase-7. Caspases play a critical role in the execution phase of apoptosis. Important targets of caspases include cytoplasmic and nuclear proteins such as keratin 18, poly ADP ribose polymerase (PARP) and lamins. Overexpression of caspase-3 induces apoptosis. Through the use of synthetic peptides, caspases have been divided into three groups based on the four amino acids amino-terminal to their cleavage site. Caspases-1, -4 and -5 prefer substrates containing the sequence WEXD (where X is variable). Caspases-2, -3 and -7 prefer the sequence DEXD. Caspases 6, 8 and 9 are the least demanding but have demonstrated a preference for cleaving of substrates containing either LEXD or VEXD. Because these sequences correspond to known cleavage sites of caspase targets, systems to study caspase cleavage activity have been developed. The measurement of caspase enzyme activity with fluorometric and colorimetric peptide substrates and the detection of caspase cleavage using antibodies to caspases allows the study of the apoptosis processes or screening of therapeutic agents which promote or prevent apoptosis. A typical assay would involve the cleavage of a fluorescent substrate peptide to quantitate activity. The substrate, DEVD-AFC, is composed of the fluorophore, AFC (7-amino-4-trifluoromethyl coumarin), and a synthetic tetrapeptide, DEVD (Asp-Glu-Val-Asp), which is the upstream amino acid sequence of the Caspase-3 cleavage site in PARP. DEVD-AFC emits blue light (• max=400 nm). Upon cleavage of the substrate by Caspase-3 or related caspases, with the excitation wavelength set to 400 nm, free AFC emits a yellow-green fluorescence (• max=505 nm) which can be quantified using a spectrofluorometer or a fluorescence microtiter plate reader. Comparison of the fluorescence of AFC from apoptotic samples with an uninduced control allows determination of the increase in caspase-3 activity.

See Jaeschke, et al. J. Immunol., 160:3480-3486 (1998); Talanian, et al., J. Biol. Chem., 272:9677-9682 (1997).

Yet another example of an apoptosis assay is the TUNEL assay. Briefly, cell death by apoptosis is characterized by DNA fragmentation in 200-250 and/or 30-50 kilobases. Further internucleosomal DNA fragmentation in 180-200 base pairs may also occur. Such characteristics have been used to distinguish apoptotic cells from normal or necrotic cells. To detect apoptotic cells, whatever the pattern of DNA fragmentation, the TUNEL (Terminal deoxynucleotidyl transferase (TdT) mediated dUTP Nick End Labeling) method is commonly utilized. One of the most easily measured features of apoptotic cells is the break-up of the genomic DNA by cellular nucleases. These DNA fragments can be extracted from apoptotic cells and result in the appearance of “DNA laddering” when the DNA is analyzed by agarose gel electrophoresis. The DNA of non-apoptotic cells, which remains largely intact, does not display this “laddering” on agarose gels during electrophoresis. The large number of DNA fragments appearing in apoptotic cells results in a multitude of 3′-hydroxyl ends in the DNA. This property can be used to identify apoptotic cells by labeling the 3′-hydroxyl ends with bromolated deoxyuridine triphosphate nucleotides (Br-dUTP). The enzyme terminal deoxynucleotidyl transferase (TdT) catalyzes a template independent addition of deoxyribonucleoside triphosphates to the 3′-hydroxyl ends of double- or single-stranded DNA with either blunt, recessed or overhanging ends. See Li and Darzynkiewicz, Cell Prolif., 28:572-579 (1995).

In another apoptosis assay, the cell death ELISA detects the same endpoint as the TUNEL assay, DNA fragmentation. However, in the cell death ELISA assay, the histone complexed DNA fragments are measured directly by antibodies in an ELISA assay. See Piro, et al., Metabolism, 51:1340-7 (2002); Facchiano et al., Exp. Cell Res., 271:118-29 (2001); Horigome et al., Immunopharmacology, 37:87-94 (1997).

The invention further provides a method of decreasing cell proliferation, comprising introducing into said cell an agent that down-modulates the activity of SEQ ID NO: 1, 2, 3, 4 or 5. The agent can be a ribozyme, an antisense molecule, an antagonizing antibody or an siRNA, as discussed above.

An example of a cell proliferation assay is the alamarBlue™ assay, which is described for instance in Example 1. Other examples include the Natural Red, methylene blue and tetrazolium/formazan assays. See also biosource.com; Alley et al., Cancer Res., 48:589-601 (1988); Elliot and Auersperg, Biotech. Histochem., 68:29-35 (1993); and Scudiero et al., Cancer Res., 48:4827-33 (1988). Another example of a cell proliferation assay is based on the cleavage of the terazolium salt WST-1. http://biochem.roche.com/pack-insert/1644807a.pdf (Roche, Version 3, May, 1999).

Another example of an assay that measures a difference in cell proliferation (or tumorigenicity) is a soft agar assay, as described in Example 1. See also Bergo et al., Mol. and Cell. Biol., 22:171-181 (2002); Zeng et al., Cancer Res., 62:3538:3543 (2002).

The invention further provides a method of identifying a molecule that inhibits cancer cells by introducing the molecule into cells, where the molecule down-modulates the RNA correlate of SEQ ID NOS:1, 2, 3, 4 or 5. The method further provides measuring the level of down-modulation of the compound, where an increase in level of down-modulation indicates that said molecule inhibits cancer cells.

Measurement of down-modulation can be made by a reporter assay using a reporter gene operably linked to a nucleic acid encoding any of the nuclear receptors of the invention. Reporter genes can express proteins such as β-lactamase, luciferase, green fluorescent protein, β-galactosidase, secreted alkaline phosphatase, human growth hormone and chlororamphenicol acetyltransferase.

The invention also provides a method of identifying a compound that inhibits cancer cells by introducing the compound into cells, where it down-modulates SEQ ID NOS:6, 7, 8, 9 or 10. The method further provides measuring the level of down-modulation of the compound, where an increase in level of down-modulation indicates that said molecule inhibits cancer cells. Down-modulation can be measured, for example, by an immunoassay using an antibody specific to said compound. Such an immunoassay can be, for example, an immunofluorescence, immunochemistry or immunoprecipitation assay.

As used herein, the term “unmodified base” means one of the bases adenine, guanine, cytosine, uracil or thymine attached to the 1-carbon of the sugar (deoxyribose or ribo-furanose), with a phosphate bound to the 5-carbon of the sugar. Bases are bound to each other via phosphodiester bonds between the 3-carbon of one base and the 5-carbon of the next base.

As used herein, the term “modified base” means any base whose chemical structure is modified as follows. Adenine can be modified to result in 6-dimethyl-amino-purine, 6-methyl-amino-purine, 2-amino-purine, 2,6-diamino-purine, 6-amino-8-bromo-purine or 6-amino-8-fluoro-purine. Cytosine can be modified to result in 5-bromo-cytosine, 5-fluoro-cytosine, N,N-dimethyl-cytosine, N-methyl-cytosine, 2-thio-cytosine or 2-pyridone. Guanine can be modified to result in 8-bromo-guanine, 8-fluoroguanine, 2-amino-purine, hypozanthine (inosine), 7-deaza-guanine or 6-thio-guanine. Uracil can be modified to result in 3-methyl-uracil, 5,6-dihydro-uracil, 4-thio-uracil, thymine, 5-bromo-uracil, 5-iodo-uracil or 5-fluoro-uracil. Thymine can be modified to result in 3-methyl-thymine, 5,6-dihydro-thymine, 4-thio-thymine, uracil, 5-bromo-uracil, 5-iodo-uracil or 5-fluoro-uracil. Methods of making such modifications as well as other modifications, such as halogen, hydroxy, amine, alkyl, azido, nitro and phenyl substitutions are disclosed in U.S. Pat. No. 5,891,684; and U.S. Pat. No. 5,298,612. The present invention encompasses sequences where one or more bases are modified.

In addition, the sugar moiety of a base can be modified as disclosed above regarding bases of a hammerhead ribozyme. The present invention encompasses sequences where one or more bases are so modified.

As used herein, the term “nucleic acid” or “nucleic acid molecule” refers to deoxyribonucleotides or ribonucleotides, oligomers and polymers thereof, in either single- or double-stranded form. Unless specifically limited, the term encompasses nucleic acids containing known analogues of natural nucleotides that have similar binding properties as the reference nucleic acid. For example, as disclosed herein, such analogues include those with substitutions, such as methoxy, at the 2-position of the sugar moiety. Unless otherwise indicated by the context, the term is used interchangeably with gene, cDNA and mRNA encoded by a gene.

As used herein, the phrase “a nucleotide sequence encoding” refers to a nucleic acid which contains sequence information, for example, for a ribozyme, mRNA, siRNA, and the like, or for the primary amino acid sequence of a specific protein or peptide. In reference to a ribozyme, unless otherwise indicated, the explicitly specified encoding nucleotide sequence also implicitly covers sequences that do not materially effect the specificity of the ribozyme for its target nucleic acid. In reference to a protein or peptide, unless otherwise indicated, the explicitly specified encoding nucleotide sequence also implicitly encompasses variations in the base sequence encoding the same amino acid sequence (e.g., degenerate codon substitutions). The invention also contemplates proteins or peptides with conservative amino acid substitutions. The identity of amino acids that may be conservatively substituted is well known to those of skill in the art. Degenerate codons of the native sequence or sequences may be chosen to conform with codon preference in a specific host cell.

As used herein, the term “RNA correlate” of a given DNA sequence means that sequence with “U” substituted for “T,” with the entire sequence in ribonucleic acid form. The present invention encompasses the RNA correlates of SEQ ID NOS:1, 2, 3, 4 and 5.

The terms “sequence similarity”, “sequence identity”, or “percent identity,” in the context of two or more nucleic acids or polypeptide sequences, refer to two or more sequences or subsequences that are, when optimally aligned with appropriate nucleotide insertions or deletions, the same or have a specified percentage of amino acid residues or nucleotides that are the same (i.e., 50% identity, 65%, 70%, 75%, 80%, preferably 85%, 90%, 91%, 92%, 93%, 94%, 95%, 96%, 97%, 98%, 99% or higher identity to an amino acid sequences such as SEQ ID NOS:6, 7, 8, 9, 10, 12, 13, 30, 32 or 34 (or ZF or HOLI domains thereof), or a nucleotide sequence such as SEQ ID NOS:1, 2, 3, 4, 5, 45, 47, 49, 51 or 53 (or RNA correlates thereof), when compared and aligned for maximum correspondence over a comparison window, or designated region as measured using one of the following sequence comparison algorithms or by manual alignment and visual inspection. This definition also refers to the complement of a test sequence. Preferably, the identity exists over a region that is at least about 25 amino acids or nucleotides in length, or more preferably over a region that is 50-100 amino acids or nucleotides in length. These relationships hold, notwithstanding evolutionary origin (Reeck et al., Cell, 50:667 (1987)). When the sequence identity of a pair of polynucleotides or polypeptides is greater or equal to 65%, the sequences are said to be “substantially identical.”

Alternatively, substantial identity will exist when a nucleic acid will hybridize under selective hybridization conditions, to a strand or its complement. Typically, selective hybridization will occur when there is at least about 55% homology over a stretch of at least about 14 nucleotides, more typically at least about 65%, preferably at least about 75%, and more preferably at least about 90%. See, Kanehisa, Nuc. Acids Res., 12:203-213 (1984), which is incorporated herein by reference. The length of homology comparison, as described, may be over longer stretches, and in certain embodiments will be over a stretch of at least about 17 nucleotides, generally at least about 20 nucleotides, ordinarily at least about 24 nucleotides, usually at least about 28 nucleotides, typically at least about 32 nucleotides, more typically at least about 40 nucleotides, preferably at least about 50 nucleotides, and more preferably at least about 75 to 100 or more nucleotides.

Amino acid sequence homology, or sequence identity, is determined by optimizing residue matches, if necessary, by introducing gaps as required. This changes when considering conservative substitutions as matches. Conservative substitutions typically include substitutions within the following groups: [glycine, alanine]; [valine, isoleucine, leucine]; [aspartic acid, glutamic acid]; [asparagine, glutamine]; [serine, threonine]; [lysine, arginine]; and [phenylalanine, tyrosine]. Homologous amino acid sequences are intended to include natural allelic and interspecies variations in each respective receptor sequence. Typical homologous proteins or peptides will have from 25-100% homology (if gaps can be introduced), to 50-100% homology (if conservative substitutions are included). Homology measures will be at least about 50%, generally at least 56%, more generally at least 62%, often at least 67%, more often at least 72%, typically at least 77%, more typically at least 82%, usually at least 86%, more usually at least 90%, preferably at least 93%, and more preferably at least 96%, and in particularly preferred embodiments, at least 98% or more.

In relation to proteins, the term “homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell, 50:667 (1987)). The present invention naturally contemplates homologues of the nuclear receptor proteins disclosed herein, and polynucleotides encoding the same, as falling within the scope of the invention.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

It should be understood that each method of the invention described herein encompasses: a) all compounds having about 80%, 85%, 90%, 92%, 95%, 96%, 97%, 98% or 99% identity with SEQ ID NOS:1 to 10 and RNA correlates of SEQ ID NOS:1-5; b) all compounds with 50, 60, 70, 80, 90 or more amino acids and having about 92%, 95%, 96%, 97%, 98% or 99% identity with SEQ ID NOS:6 to 10; c) all compounds having about 80%, 85%, 90%, 92%, 95%, 96%, 97%, 98% or 99% identity with all ZF and HOLI domains of SEQ ID NOS:6 to 10; and c) all compounds with 100, 150, 200, 250, 300 or more nucleotides and having about 92%, 95%, 96%, 97%, 98% or 99% identity with SEQ ID NOS:1 to 5 or their RNA correlates. The invention also encompasses all encoding DNA of SEQ ID NOS:6 to 10, as well as RNA correlates of such DNA.

The term “moderately stringent conditions,” as used herein, means hybridization conditions that permit a nucleic acid molecule to bind to a second nucleic acid molecule that has substantial identity to the sequence of the first. Moderately stringent conditions are those equivalent to hybridization of filer-bound nucleic acid in 50% formamide, 5× Denhart's solution, 5×SSPE, 0.2% SDS at 42° C., followed by washing in 0.2×SSPE, 0.2% SDS at 50° C. “Highly stringent conditions” are those equivalent to hybridization of filer-bound nucleic acid in 50% formamide, 5× Denhart's solution, 5×SSPE, 0.2% SDS at 42° C., followed by washing in 0.2×SSPE, 0.2% SDS at 65° C. Other suitable moderately stringent and highly stringent conditions are known in the art and described, for example, in Sambrook et al., Molecular Cloning: A Laboratory Manual, Cold Spring Harbor Laboratory, New York (1992), and Ansubel et al., Current Protocols in Molecular Biology, John Wiley and Sons, Baltimore Md. (1998).

In general, a nucleic acid molecule that hybridizes to a second one under moderately stringent conditions will have greater than about 60% identity, preferably greater than about 70% identity and, more preferably, greater than about 80% identity over the length of the two sequences being compared. A nucleic acid molecule that hybridizes to a second one under highly stringent conditions will have greater than about 90% identity, preferably greater than about 92% identity and, more preferably, greater than about 95%, 96%, 97%, 98% or 99% identity over the length of the two sequences being compared.

As used herein, the term “isolated” when used in conjunction with a nucleic acid or protein, denotes that the nucleic acid or protein has been isolated with respect to the many other cellular components with which it is normally associated in the natural state. For example, an “isolated” gene of interest may be one that has been separated from open reading frames which flank the gene and encode a gene product other than that of the specific gene of interest. Such genes may be obtained by a number of methods including, for example, laboratory synthesis, restriction enzyme digestion or PCR. Likewise, an “isolated” protein may be substantially purified from a natural source or may be synthesized in the laboratory. A “substantially purified” nucleic acid or protein gives rise to essentially one band in an electrophoretic gel, and is at least 85% pure, more preferably at least 95% pure, and most preferably at least 99% pure.

The term “intrabody” refers to a class of neutralizing molecules with applications in gene therapy (vonMehren M, Weiner L M. (1996) Current Opinion in Oncology. 8:493-498, Marasco Wash. (1997) Gene Therapy. 4:11-15, Rondon I J, Marasco Wash. (1997) Annual Review of Microbiology. 51:257-283). Intrabodies are engineered antibodies that can be expressed within a cell and target an intracellular molecule. Using this technique, intracellular signals and enzyme activities can be inhibited, or their transport to cellular compartments prevented. Marasco, W. A., et al., Proc. Natl. Acad. Sci. USA 90:7889-7893 (1993). Thus, intrabodies provide yet another approach to down regulating nuclear receptor expression and activity.

The intrabody method is analogous to the inactivation of proteins by deletion or mutation, but is directed at the level of gene product rather than at the gene itself. Using the intrabody strategy even molecules involved in essential cellular pathways can be targeted, modified or blocked. Antibody genes for intracellular expression can be derived either from murine or human monoclonal antibodies or from phage display libraries. For intracellular expression small recombinant antibody fragments, containing the antigen recognizing and binding regions, can be used. Intrabodies can be directed to different intracellular compartments by targeting sequences attached to the antibody fragments. The construction and use of intrabodies is discussed, for example, in U.S. Pat. No. 6,004,940.

As used herein, the term “expression vector” includes a recombinant expression cassette that has a nucleotide sequence that can be transcribed into RNA in a cell. The cell can further translate transcribed mRNA into protein. An expression vector can be a plasmid, virus, or nucleic acid fragment. Typically, the recombinant expression cassette portion of an expression vector includes the encoding nucleotide sequence to be transcribed (e.g. a ribozyme or siRNA), operably linked to a promoter, or other regulatory sequence by a functional linkage in cis. In accordance with the present invention, an expression vector comprising a nucleotide sequence encoding ribozymes of the invention can be used to transduce cells suitable as hosts for the vector. Both procaryotic cells including bacterial cells such as E. coli and eukaryotic cells including mammalian cells may be used for this purpose.

As used herein, the term “promoter” includes nucleic acid sequences near the start site of transcription (such as a polymerase binding site) and, optionally, distal enhancer or repressor elements (which may be located several thousand base pairs from the start site of transcription) that direct transcription of the nucleotide sequence in a cell. The term includes both a “constitutive” promoter such as a pol III promoter, which is active under most environmental conditions and stages of development or cell differentiation, and an “inducible” promoter, which initiates transcription in response to an extracellular stimulus, such as a particular temperature shift or exposure to a specific chemical. Promoters and other regulatory elements (e.g., an origin of replication), and/or chromosome integration elements such as retroviral long terminal repeats (“LTRs”), or adeno associated viral (AAV) inverted terminal repeats (“ITRs”), may be incorporated into an expression vector encoding ribozymes for use in accordance with the present invention as described in WO 00/05415 to Barber et al.

As used herein, the term “expresses” denotes that a given nucleic acid comprising an open reading frame is transcribed to produce an RNA molecule. It also denotes that a given nucleic acid is transcribed and translated to produce a polypeptide. Although the term may be used to refer to the transcription of a ribozyme, a ribozyme typically is not translated into a protein since it functions as an active (catalytic) nucleic acid.

As used herein, the term “gene product” refers either to the RNA produced by transcription of a given nucleic acid or to the polypeptide produced by translation of a given nucleic acid.

As used herein, the term “transduce” denotes the introduction of an exogenous nucleic acid molecule (e.g., by means of an expression vector) inside the membrane of a cell. Exogenous DNA may or may not be integrated (covalently linked) into chromosomal DNA making up the genome of the cell. The exogenous DNA may be maintained on an episomal element, such as a plasmid. In eukaryotic cells, a stably transduced cell is generally one in which the exogenous DNA has become integrated into the chromosome so that it is inherited by daughter cells through chromosome replication, or one which includes stably maintained extrachromosomal plasmids. This stability is demonstrated by the ability of the eukaryotic cell to establish cell lines or clones comprised of a population of daughter cells containing the exogenous DNA.

The term “transfection,” as used herein, means the genetic modification of a cell by uptake of an exogenous nucleic acid molecule (e.g., by means of an expression vector).

As used herein, the term “ribozyme gene vector library” denotes a collection of ribozyme-encoding genes, typically within expression cassettes, in a collection of viral or other vectors. The vectors may be naked or contained within a capsid. Propagation of the ribozyme gene vector library can be performed as described in WO 00/05415 to Barber et al. The ribozyme-encoding genes of a ribozyme gene vector library, after transduction and transcription in appropriate cells, produce a collection of ribozymes.

As used herein, “small interfering RNAs” (siRNA) are short double-stranded RNA fragments that elicit a process known as “RNA interference” (RNAi), a form of sequence-specific gene silencing. Zamore, Phillip et al., Cell 101:25-33(2000); Elbashir, Sayda M., et al., Nature 411:494-497 (2001). SiRNAs are assembled into a multi-component complex known as the RNA-induced silencing complex (RISC). The siRNAs guide RISC to homologous mRNAs, thus targeting them for destruction. Hammond et al., Nature Genetics Reviews 2:110-119(2000). RNAi has been observed in a variety of organisms including plants, insects and mammals, and cultured cells derived from these organisms.

An “siRNA” is a double-stranded RNA that is preferably between 16 and 25, more preferably 17 and 23 and most preferably between 18 and 21 base pairs long, each strand of which has a 3′ overhang of 2 or more nucleotides. Functionally, the characteristic distinguishing an siRNA over other forms of dsRNA is that the siRNA comprises a sequence capable of specifically inhibiting genetic expression of a gene or closely related family of genes by a process termed RNA interference.

siRNAs for use in the present invention can be produced from a nuclear receptor encoding nucleic acid sequence (SEQ ID NOS:1-5). For example, short complementary DNA strands are first prepared that represent portions of both the “sense” and “antisense” strands of the NR4A1 coding region. This is typically accomplished using solid phase nucleic acid synthesis techniques, as known in the art. The short duplex DNA thus formed is ligated into a suitable vector that is then used to transfect a suitable cell line. Other methods for producing siRNA molecules are known in the art. (See, e.g., Elbashir, S. M., Harborth, J., Lendeckel, W., Yalcin, A., Weber, K., and Tuschl, T. (2001). For a review of RNAi and siRNA expression, see Hammond, Scott M. et al., Nature Genetics Reviews, 2:110-119; Fire, Andrew (1999) TIG, 15(9):358-363; Bass, Brenda L. (2000) Cell, 101:235-238. An siRNA of the invention can be constructed using, for example, a Lentiviral vector for stable expression.

siRNA molecules can be transfected into a cell line, for example HeLa, by using an agent such as Oligofectamine™, as described in Example 1. See also Invitrogen Corp., Transfecting siRNA into HeLa Cells Using Oligofectamine™, Doc. Rev. 102902 (Carlsbad, Calif.); Elbashir, et al., Nature, 411:494-498 (2001); and Harborth et al., Science, 114:4557-4565 (2001).

The targeting of antisense oligonucleotides to mRNA is another mechanism of decreasing protein synthesis, and, consequently, represents a powerful and targeted approach to diminishing expression of the nuclear receptors of the invention. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829, each specifically incorporated herein by reference in its entirety). Further, examples of antisense inhibition have been demonstrated with the nuclear protein cyclin, the multiple drug resistance gene (MDG1), ICAM-1, E-selectin, STK-1, striatal GABA.sub.A receptor and human EGF (Jaskulski et al., 1988; Vasanthakumar and Ahmed, 1989; Peris et al., 1998; U.S. Pat. Nos. 5,801,154; 5,789,573; 5,718,709 and 5,610,288, each specifically incorporated herein by reference in its entirety). Antisense constructs have also been described that inhibit and can be used to treat a variety of abnormal cellular proliferations, e.g. cancer (U.S. Pat. Nos. 5,747,470; 5,591,317 and 5,783,683, each specifically incorporated herein by reference in its entirety).

The invention provides therefore oligonucleotide sequences that comprise all, or a portion of, any sequence that is capable of specifically binding to a polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of SEQ ID NOS:1, 2, 3, 4 or 5.

Selection of antisense compositions specific for a given gene sequence is based upon analysis of the chosen target sequence and determination of secondary structure, binding energy, and relative stability. Antisense compositions are selected based upon their relative inability to form dimers, hairpins, or other secondary structures that would reduce or prohibit specific binding to the target mRNA in a host cell.

“Non-native promoter” refers to any promoter element operably linked to a coding sequence by recombinant methods. Non-native promoters include mutagenized native reporters, when mutagenesis alters the rate or control of transcriptional events.

“Operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence.

The invention also encompasses vectors in which a nuclear receptor nucleic acid is cloned into a vector in reverse orientation, but operably linked to a regulatory sequence that permits transcription of antisense RNA. Thus, an antisense transcript can be produced to all, or to a portion, of the nucleic acid sequences described herein, including both coding and non-coding regions. Expression of this antisense RNA is subject to each of the parameters described above in relation to expression of the sense RNA (regulatory sequences, constitutive or inducible expression, tissue-specific expression).

Antisense nucleic acids may be obtained from libraries encoding nuclear receptor proteins of the invention (SEQ ID NOS:6-10) or synthesized synthetically. Transfection of suitable host cells with such a protein is performed in a manner analogous to that described for siRNAs above.

“Recombinant expression cassette” refers to a DNA sequence capable of directing expression of a nucleic acid in cells. A “DNA expression cassette” comprises a promoter, operably linked to a nucleic acid of interest, which is further operably linked to a termination region.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Candidate protein-based compounds for binding or down-modulating a nuclear receptor protein of the invention or one of its ZF or HOLI domains include, for example, 1) peptides such as soluble peptides, including fusion peptides and members of random peptide libraries (see, e.g., Lam et al., Nature 354:82-84 (1991); Houghten et al., Nature 354:84-86 (1991)) and combinatorial chemistry-derived molecular libraries made of L- and/or D-configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies, including intrabodies, as well as Fab, F(ab′).sub.2, Fab expression library fragments, and epitope-binding fragments of antibodies); and 4) small organic and inorganic molecules (e.g., molecules obtained from combinatorial and natural product libraries).

Soluble full-length receptors, or fragments of the same, that compete for ligand binding are also considered candidate reagents. Other candidate compounds include mutant receptors or appropriate fragments containing mutations that affect receptor function and thus compete for ligand. Accordingly, a fragment that competes for ligand, for example with a higher affinity, or a fragment that binds ligand but does not allow release, is encompassed by the invention. The receptor polynucleotides are also useful for constructing host cells expressing a part, or all, of the receptor polynucleotides and polypeptides.

The receptor polynucleotides are also useful for constructing transgenic animals expressing all, or a part, of the receptor polynucleotides and polypeptides. These animals are useful as model systems for the treatment of cancer and can be used to test compounds for their effect, through the receptor gene or gene product, on the development or progression of the disease.

As used herein, the term “knockdown” or “down-modulation” means a decrease in the rate or level of mRNA production and/or protein production. For example, siRNA constructs of the present invention were shown to down-modulate the mRNA of the target genes of the invention (SEQ ID NOS:1-5), as discussed above and in the examples below.

The present invention also provides a method of inhibiting cancer cells or their growth. This method includes introducing an agent of the invention into cancer cells. Such an agent can be, for example, a ribozyme of the present invention, an antisense molecule, an antagonizing antibody or an siRNA. This method can comprise transducing the infected cell with an expression vector encoding the agent. Alternatively, the agent can be introduced into a cell directly, i.e., without using a vector.

The method of the invention can be accomplished by the agent binding to a target, for instance SEQ ID NOS: 1 to 10, or portions or domains thereof, as discussed above. The method of the invention can also be accomplished by the agent down-modulating SEQ ID NOS:1 to 10, or portions or domains thereof, as discussed above.

In accordance with another embodiment of the present invention, the step of down-modulating the level of the target protein in the cell can be accomplished by introducing into the cell system an antisense compound or molecule.

Combinatorial peptide libraries can be screened to identify antagonists of a protein, ZF domain or HOLI domain of the invention, which can increase the inhibition of cancer cells. Combinatorial peptide libraries can be constructed from genomic or cDNA libraries, or by using non-cellular synthetic methods. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., J. Am. Chem. Soc. 85: 2149-2156 (1963), and Stewart et al., Solid Phase Peptide Synthesis, 2nd ed., Pierce Chem. Co., Rockford, III. (1984). Proteins may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill.

The proteins useful in this invention may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503.

Peptide and protein reagents can optionally be labeled, as described below, or may be used in the screening assays of the present invention to ascertain their ability to modulate nuclear receptor expression or activity.

Portions of SEQ ID NOS:6-10 can be useful in competition binding assays in methods designed to discover compounds that interact with the receptor. Thus, a compound is exposed to a receptor polypeptide under conditions that allow the compound to bind or to otherwise interact with the polypeptide. Soluble receptor polypeptide is also added to the mixture. If the test compound interacts with the soluble receptor polypeptide, it decreases the amount of complex formed or activity from the receptor target. This type of assay is particularly useful in cases in which compounds are sought that interact with specific regions of the receptor. Thus, the soluble polypeptide that competes with the target receptor region is designed to contain peptide sequences corresponding to the region of interest.

The compounds tested as modulators of proteins or protein domains of the invention or RNA correlates of the invention can be any small chemical compound, or a biological entity, such as a protein, sugar, nucleic acid or lipid. Screening combinatorial libraries of small organic molecules offers an approach to identifying useful therapeutic compounds or precursors targeted to proteins or protein domains of the invention or RNA correlates of the invention. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods are utilized involving a combinatorial chemical or peptide library containing a large number of potential therapeutic compounds (potential modulator or ligand compounds). Such “combinatorial chemical libraries” or “ligand libraries” are screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. Preparation and screening of combinatorial chemical libraries is well known to those of skill in the art.

To perform cell-free drug screening assays, it is desirable to immobilize either the receptor protein, or fragment, or its target molecule to facilitate separation of complexes from uncomplexed forms of one or both of the proteins, as well as to accommodate automation of the assay.

In one embodiment, the invention provides soluble assays using molecules such as a ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; a nuclear receptor protein of the invention; or a cell or tissue expressing such protein, either naturally occurring or recombinant. In another embodiment, the invention provides solid phase based in vitro assays in a high throughput format, where the domain, chimeric molecule, protein of the invention, or cell or tissue expressing such protein is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed, e.g., by Caliper Technologies (Palo Alto, Calif.).

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available as are appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Another approach uses recombinant bacteriophage to produce large libraries. Using the “phage method” (Scott and Smith, Science 249:386-390, 1990; Cwirla, et al, Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al, Proc. Natl. Acad. Sci. USA 90:10700-4, 1993; Ohlmeyer et al., Proc. Natl. Acad. Sci. USA 90:10922-10926, 1993; Lam et al., International Patent Publication No. WO 92/00252; Kocis et al., International Patent Publication No. WO 9428028) and the like can be used to screen for a protein or domain according to the present invention.

The screening can be performed with recombinant cells that express the protein or domain of the invention, or alternatively, using purified protein, e.g., produced recombinantly, as described above. For example, the ability of labeled, soluble or solubilized NR4A1 that includes the ligand-binding portion of the molecule, to bind ligand can be used.

Radioligand binding assays allow further characterization of hits from high throughput screens as well as analogs of neurotensin agonists and antagonists. Using membranes from cells stably expressing each neurotensin receptor subtype, one point binding assays are first performed to determine how well a particular concentration of each hit or analog displaces specific [³H] NT binding from the receptor. If the hit or analog displaces ≧50% of the [³H] NT bound, a competition binding assay is performed. Competition binding assays can evaluate the ability of increasing concentrations of competitor (the hit or any test compound analog) to displace [3H] NT binding at each neurotensin receptor subtype. The resulting Kᵢ value indicates the relative potency of each hit or test compound for a particular receptor subtype. These competition binding assays allow the determination of the relative potencies of each hit or test compound at a particular receptor subtype, as well as to determine the receptor subtype selectivity of each hit or test compound.

Yet another assay for compounds that modulate nuclear receptor activity involves computer assisted drug design, in which a computer system is used to generate a three-dimensional structure of a protein of the invention based on the structural information encoded by the amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a nuclear receptor polypeptide into the computer system. Contiguous portions of SEQ ID NOS:1-5, and conservatively modified versions thereof, can be used for this purpose. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program therefore uses these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the nuclear receptor protein to identify ligands that bind to such protein. Binding affinity between the protein and ligands is determined using energy terms to determine which ligands have an enhanced probability of binding to the protein.

The activity of nuclear receptor polypeptides can be assessed using a variety of in vitro and in vivo assays that determine functional, physical and chemical effects, e.g., measuring ligand binding (e.g., by radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of proteins or domains of the invention. Modulators can also be genetically altered versions of nuclear receptors. Such modulators can be useful in the treatment and diagnosis of cancer.

The polypeptide of the assay will be selected from SEQ ID NOS:6-10, a portion of 10, 20, 30, 40, 50 or more contiguous amino acids thereof, or conservatively modified variants thereof. Alternatively, the nuclear receptor protein of the assay will be derived from a eukaryote and include an amino acid subsequence where the homology will be at least 60%, preferably at least 75%, more preferably at least 90% and most preferably between 95% and 100% that of SEQ ID NOS:6-10. Optionally, the polypeptide of the assays will comprise a domain of SEQ ID NOS:6-10. Either SEQ ID NOS:6-10 or a domain thereof can be covalently linked to a heterologous protein to create a chimeric protein used in the assays described herein.

Modulators of nuclear receptor protein activity are tested using polypeptides as described above, either recombinant or naturally occurring. The protein can be isolated, expressed in a cell, expressed in a membrane derived from a cell, expressed in tissue or in an animal, either recombinant or naturally occurring.

Changes in ion flux may be assessed by determining changes in polarization (i.e., electrical potential) of the cell or membrane expressing a nuclear receptor protein. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J. Membrane Biology 137:59-70 (1994)). Generally, the compounds to be tested are present in the range from 1 pM to 100 mM.

The practice of using a reporter gene to analyze nucleotide sequences that regulate transcription of a gene-of-interest is well documented. In particular, the promoters of the genes of the present invention, i.e., SEQ ID NOS:1-5, contain receptor responsive elements. These elements can confer a particular effect of the transcription factor nuclear hormone receptor of the invention and can be read by a reporter system, as described below.

The demonstrated utility of a reporter gene is in its ability to define domains of transcriptional regulatory elements of a gene-of-interest. Reporter genes express proteins that serve as detectable labels indicating when the control elements regulating reporter gene expression are up or down-regulated in response to outside stimuli.

By way of example, two types of reporter gene assay are discussed below. The first is a scorable reporter gene, whose expression can be quantified, giving a proportional indication of the level of expression supported by the genetic construct comprising the reporter gene. The second example is a selectable reporter gene. When expressed, the selectable reporter gene allows the host cell harboring the reporter gene to survive under restrictive conditions that would otherwise kill (or retard the growth of) the host cell.

Scorable reporter genes are typically used when the relative activity of a genetic construct is sought, whereas selectable reporters are used when confirmation of the presence of the reporter expression construct within the cell is desired.

Firefly luciferase expression systems have become widely used for quantitative analysis of transcriptional modulation in living cells (see, e.g., Wood, K. V. (1998) Promega Notes 65:14). In particular, recombinant cells comprising this reporter construct enable libraries of small molecules to be rapidly screened for those affecting specific aspects of cellular physiology, such as receptor function or intracellular signal transduction. deWet et al. (1987) Mol. Cell. Biol. 7:725; Wood, K. V. (1991) In: Bioluminescence and Chemiluminescence: Current Status, eds. P. Stanley and L. Kricka, John Wiley and Sons, Chichester, 11.

The luciferase assay could be used to screen any of the potential reagents listed above. For example, by placing the luciferase gene under the control of the NR4A1 promoter, reagents that bind to the NR4A1 protein can trigger a feedback loop modulating expression of the luciferase gene. Similarly, by creating a fusion protein comprising the luciferase and NR4A1 coding sequences, siRNAs, antisense sequences and ribozymes targeted against the NR4A1 gene can be screened, as any reagent acting on the NR4A1 transcript will necessarily disrupt expression of the luciferase enzyme encoded in the same transcript.

Modulators will manifest themselves by altering the amount of light emitted by the luciferase-catalyzed hydrolysis of ATP, with up-modulators increasing the amount of light emitted (they induce increased luciferase production) and down-modulators decreasing the amount of light emitted (by inhibiting luciferase production) in proportion to the degree of expressional modulation (at least within the linear range limits of the assay). Luciferase assay kits and other reporter gene constructs suitable for use in the present invention are well known in the art and commercially available, e.g., Invitrogen and Promega. See, e.g., Steady-Glo™ Luciferase Assay Reagent Technical Manual Luciferase Assay Reagent Technical Manual #TM051, Promega Corporation.

As an example of a reporter gene assay, the NurRE from the NR4A1 target POMC can be inserted in front of the SV40 promoter in a pGL3-promoter construct (Promega) so that the luciferase can be induced by NR4A1 expression. Down-regulation of NR4A1 can be assayed to determine whether the NR4A1 protein level correlates to its ability to induce reporter gene expression. For further description of reporter system assays, see, for example, Unit 9.6 of Current Protocols in Molecular Biology, (John Wiley & Sons, 1997).

A number of selectable marker systems can be used in the present invention, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Typically, selectable markers are included in expression cassettes comprising the target gene or construct to be incorporated into the host cell. The selectable marker may be under the control of the same promoter as the target construct, e.g., as part of a fusion protein or polycistronic transcript; or may be under the control of an independent promoter.

As suggested above, the purpose of the selectable marker is to confer selectable growth characteristics on cells that are able to express it. By including the selectable marker in the same nucleic acid comprising the target gene or construct, the selectable marker will be included in any cell transformed with the target. Therefore, by selecting for the growth characteristics conferred by the selectable marker, cells transfected with the target can be selected.

Real-time PCR assays, as described in the examples herein, take advantage of those cycles of a normal PCR reaction where the DNA being amplified is increasing at a logarithmic rate and hence proportional to the amount of DNA present. Several kits are commercially available for performing real-time PCR. One such kit is the TaqMan® assay.

The TaqMan® assay takes advantage of the 5′ nuclease activity of Taq DNA polymerase to digest a DNA probe annealed specifically to the accumulating amplification product. TaqMan® probes are labeled with a donor-acceptor dye pair that interacts via fluorescence energy transfer. Cleavage of the TaqMan® probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time. In an alternative homogeneous hybridization based procedure, molecular beacons are used for allele discriminations. Molecular beacons are hairpin-shaped oligonucleotide probes that report the presence of specific nucleic acid molecules in homogeneous solutions. When they bind to their targets they undergo a conformational reorganization that restores the fluorescence of an internally quenched fluorophore. (See, e.g., Heid, C. A., Stevens, J., Livak, K. J. and Williams, P. M. Real time quantitative PCR. Genome Res. 6:986-994 (1996); Gibson, U. E. M., Heid, C. A. and Williams, P. M. A novel method for real time quantitative RT-PCR. Genome Res. 6:995-1001 (1996)).

Northern blot methods allow RNA isolated from cells of interest to be separated using gel electrophoresis techniques. After separation, nucleic acids are transferred to membranes and hybridized with radio-labeled nucleotide probes. For analysis of expression maps, poly A (adenylyl) probed are used, which hybridize to mRNA species present on the blot.

The present invention includes both traditional and expression map Northern blotting. Expression of SEQ ID NOS:1-5 and other genes of interest can be tracked using probes specific for these genes. Expression mapping can be used to monitor alterations in gene expression in response to nuclear receptor protein-specific binding agents.

Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996.

Through the use of high density oligonucleotide arrays, expression profiles for individual cells can be rapidly obtained and compared. High density arrays are particularly useful for monitoring expression control at the transcriptional, RNA processing and degradation level. The fabrication and application of high density arrays in gene expression monitoring have been disclosed in, for example, WO 97/10365, WO 92/10588, U.S. Pat. No. 6,040,138 incorporated herein for all purposes by reference. In some embodiments using high density arrays, high density oligonucleotide arrays are synthesized using methods such as the Very Large Scale Immobilized Polymer Synthesis (VLSIPS) disclosed in U.S. Pat. No. 5,445,934. Each oligonucleotide occupies a known location on a substrate. A nucleic acid target sample is hybridized with a high density array of oligonucleotides and then the amount of target nucleic acids hybridized to each probe in the array is quantified. One preferred quantifying method is to use confocal microscope and fluorescent labels. The GeneChip.RTM. system (Affymetrix, Santa Clara, Calif.) is particularly suitable for quantifying the hybridization; however, it will be apparent to those of skill in the art that any similar systems or other effectively equivalent detection methods can also be used.

High density arrays are suitable for quantifying a small variations in expression levels of a gene in the presence of a large population of heterogeneous nucleic acids. Such high density arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. Suitable nucleic acids are also produced by amplification of templates. As a nonlimiting illustration, polymerase chain reaction, and/or in vitro transcription, are suitable nucleic acid amplification methods.

Synthesized oligonucleotide arrays are particularly preferred for this invention. Oligonucleotide arrays have numerous advantages, as opposed to other methods, such as efficiency of production, reduced intra- and inter array variability, increased information content and high signal-to-noise ratio.

An “antisense compound or molecule” refers to such compound or molecule that includes a polynucleotide that is complementary to a target sequence of choice and capable of specifically hybridizing with the target molecules. The term antisense includes a “ribozyme,” which is a catalytic RNA molecule that cleaves a target RNA through ribonuclease activity. Antisense nucleic acids hybridize to a target polynucleotide and interfere with the transcription, processing, translation or other activity of the target polynucleotide. An antisense nucleic acid can inhibit DNA replication or DNA transcription by, for example, interfering with the attachment of DNA or RNA polymerase to the promoter by binding to a transcriptional initiation site or a template. It can interfere with processing of mRNA, poly(A) addition to mRNA or translation of mRNA by, for example, binding to regions of the RNA transcript such as the ribosome binding site. It can promote inhibitory mechanisms of the cells, such as promoting RNA degradation via RNase action. The inhibitory polynucleotide can bind to the major groove of the duplex DNA to form a triple helical or “triplex” structure. Methods of inhibition using antisense polynucleotides therefore encompass a number of different approaches to altering expression of specific genes that operate by different mechanisms (see, e.g., Helene & Toulme, Biochim. Biophys. Acta., 1049:99-125 (1990)).

The antisense compounds that may be used in connection with this embodiment of the present invention preferably comprise between about 8 to about 30 nucleobases (i.e., from about 8 to about 30 linked nucleosides), more preferably from about 12 to about 25 nucleobases, and may be linear or circular in configuration. They may include oligonucleotides containing modified backbones or non-natural internucleoside linkages. Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Methods of preparing antisense compounds are well known in the art (see, for example, U.S. Pat. No. 6,210,892).

The present invention also provides a method of increasing inhibition of cancer cells, the method comprising introducing into the cell an effective amount of an expression vector comprising a sequence of nucleotides that encodes a ribozyme having a substrate binding sequence disclosed herein or an siRNA. The expression vector is preferably administered in combination with a suitable carrier. After the vector has been administered, the ribozyme or siRNA is expressed in the cell.

This method can be applied to a subject with cancer. Administration of the vector into the subject can be by any suitable route including oral, sublingual intravenous, subcutaneous, transcutaneous, intramuscular, intracutaneous, and the like. Any of a variety of non-toxic, pharmaceutically acceptable carriers can be used for formulation including, glucose, lactose, gum acacia, gelatin, mannitol, starch paste, magnesium trisilicate, talc, corn starch, keratin, colloidal silica, potato starch, urea, dextrans, and the like. The formulated material may take any of various forms such as injectable solutions, sterile aqueous or non-aqueous solutions, suspensions or emulsions, tablets, capsules, and the like.

As used herein, the phrase “effective amount” refers to a dose of the deliverable sufficient to provide circulating concentrations high enough to impart a beneficial effect on the recipient, which is an increase of inhibition of cancer cells.

The specific therapeutically effective dose level for any particular subject and deliverable depends upon a variety of factors including the severity of the infection, the activity of the specific compound administered, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically range from about 0.001 up to 100 mg/kg/day; with levels in the range of about 0.05 up to 10 mg/kg/day.

The present invention also provides an antibody with binding specificity for a nuclear receptor protein of the invention, such as SEQ ID NOS:6-10, or any molecule with 80%, 85%, 90% or 95% or more sequence identity with SEQ ID NOS:6-10, or any fragment of 10 or more contiguous amino acids of SEQ ID NOS:6-10. The antibody can have a binding specificity for a protein or peptide (i.e., amino acid sequence) encoded by SEQ ID NOS:1-5, or any molecule with 80%, 85%, 90% or 95% or more sequence identity with SEQ ID NOS:1-5.

As used herein, the term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies can exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology.

Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Methods of producing polyclonal and monoclonal antibodies are known to those of skill in the art. See, e.g., Coligan (1991) Current Protocols in Immunology Wiley/Greene, NY; and Harlow and Lane (1989) Antibodies: A Laboratory Manual, Cold Spring Harbor Press, NY; Stites et al. (eds.) Basic and Clinical Immunology (4th ed.) Lange Medical Publications, Los Altos, Calif.; Huse et al. (1989) Science 246:1275-1281; and Ward et al. (1989) Nature 341:544-546, and references cited therein. Techniques for the production of single chain antibodies (U.S. Pat. No. 4,946,778) can be adapted to produce antibodies to polypeptides of this invention.

Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

Methods of producing polyclonal and monoclonal antibodies that react specifically with a nuclear receptor protein of the invention are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of nuclear receptor proteins comprising immunogens may be used to produce antibodies specifically reactive with SEQ ID NOS:6-10, or portions thereof. For example, recombinant NRA41 or an antigenic fragment thereof can be isolated, as is known in the art. Recombinant protein can be expressed in eukaryotic or prokaryotic cells as described above, and purified as generally described above. Recombinant protein is one embodiment of an immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the nuclear receptor protein. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281(1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-nuclear receptor proteins of the invention or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once specific antibodies are available, the nuclear receptor proteins of the invention can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The antibodies are also useful for inhibiting receptor function, for example, blocking ligand binding. These uses can also be applied in a therapeutic context in which treatment involves inhibiting receptor function. An antibody can be used, for example, to block ligand binding. Antibodies can be prepared against specific fragments containing sites required for function or against intact receptor associated with a cell.

As used herein, the phrase “binding specificity,” in relationship to an antibody that binds to a protein or peptide, refers to a binding reaction which is determinative of the presence of the protein in the presence of a heterogeneous population of proteins and other biologics. Thus, under designated immunoassay conditions, the specified antibody binds to a particular protein and does not bind significantly to other proteins present in the sample.

The following examples are intended to illustrate but not limit the present invention.

### EXAMPLE 1

This example shows that knockdown of the nuclear receptor NR4A1 (SEQ ID NO:1; Accession #: NM_{—}002135.1) inhibits growth and/or proliferation of cancer cells.

1. General.

NR4A1, also named NGFI-B (Nerve growth factor induced clone B), Nur77, TR3, and NAK-1, is a transcription factor belonging to the superfamily of nuclear receptors (NRs). It is closely related to NR4A2 (Nurr1, TINUR, HZF-3, RNR-1) and NR4A3 (NOR1, MINOR), together forming the (Nur factor) subfamily of the orphan receptors belonging to the steroid thyroid hormone retinoid receptors. Law et al., Mol. Endocrinol., 6:2129-2135 (1992); Ohkura et al., Biochem. Biophys. Res. Commun., 205:1959-1965 (1994).

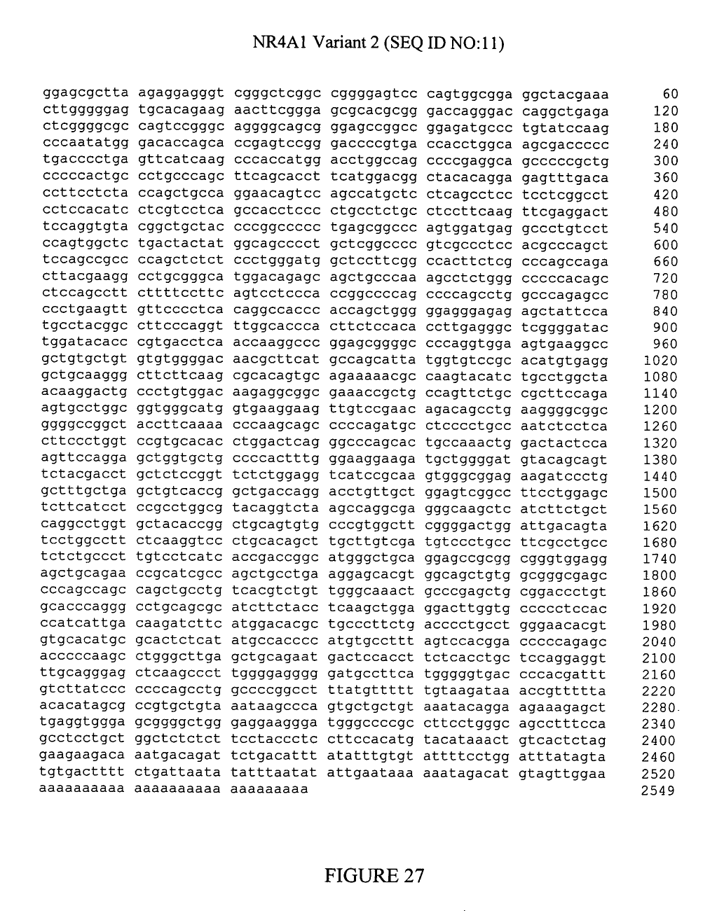

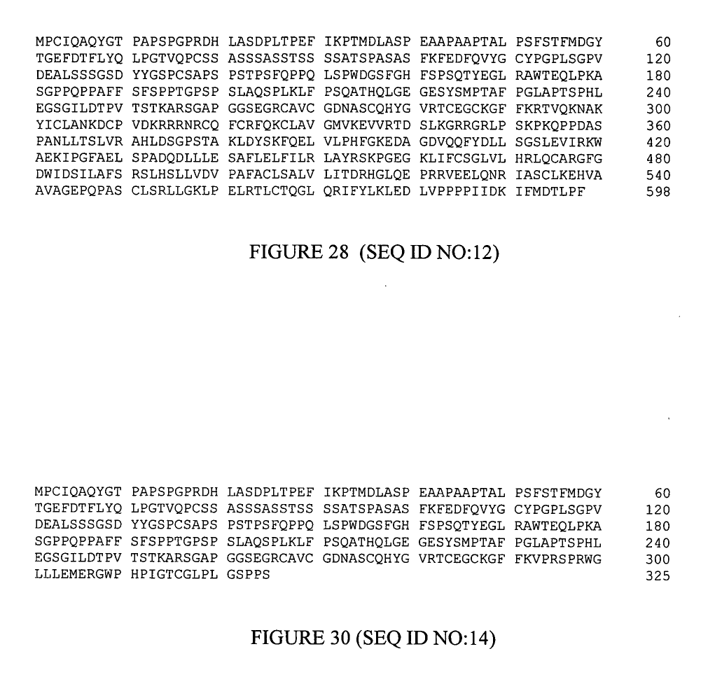

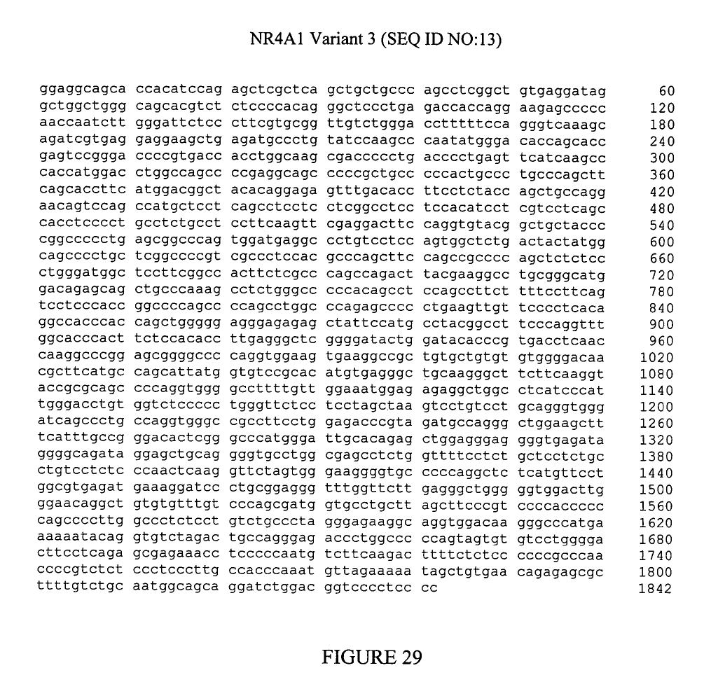

NR4A1 is expressed in various peripheral tissues and some regions of the brain. Wastson et al., Development, 110: 173-183 (1990). There are three transcript variants for NR4A1. Variant 1 (SEQ ID NO:1; NM_{—}002135) is the longest with 2699 bp. Variant 2 (SEQ ID NO:11; FIG. 27; NM_{—}173157) is identical to variant 1, with the exception of a 151 bp deletion in the 5′ UTR (2549 bp). Variant 3 (SEQ ID NO:13; FIG. 29; NM_{—}173158) is the shortest with 1842 bp, and shares 878 bp identity with variant 1. Amino acid sequence comparison of the three variants shows that variant 1 (SEQ ID NO:6) and variant 2 (SEQ ID NO:12; FIG. 28) are identical in sequence (598 AA), while variant 3 (SEQ ID NO:14; FIG. 30) is identical to variants 1 and 2 in the N-terminal 292 AA. The present invention encompasses all such variant DNA sequences, all RNA correlates thereof and all variant amino acid sequences with respect to all methods described herein.

NR4A1 has been shown to play an important role in regulating the expression of various genes in the hypothalamic-pituitary-adrenal axis (HPA) related to inflammation and steroidogenesis. Stocco, et al., J. Biol. Chem., 277: 3293-3302 (2002). Targets of NR4A1 include proopiomelanocortin (POMC), steroid-21a-hydroxylase, steroid 17-hydroxylase, 20a-hydroxysteroid dehydrogenase and corticotropin-releasing hormone (CRH) (Stocco, supra.). NR4A1 is an important regulator of apoptosis in self-reactive immature thymocytes following stimulation of the T-cell receptor (TCR) (Cheng, supra.). It has also been shown to translocate to mitochondria and induce cytochrome C release and apoptosis in LNCaP human prostate cancer cells in response to apoptotic stimuli. Li, et al., Science, 289:11 (2000). However, the apoptosis-inducing activity of NR4A1 had not previously been observed in cell lines other than LNCaP and T cells.

2. Expression of NR4A1 in Cancer Cells

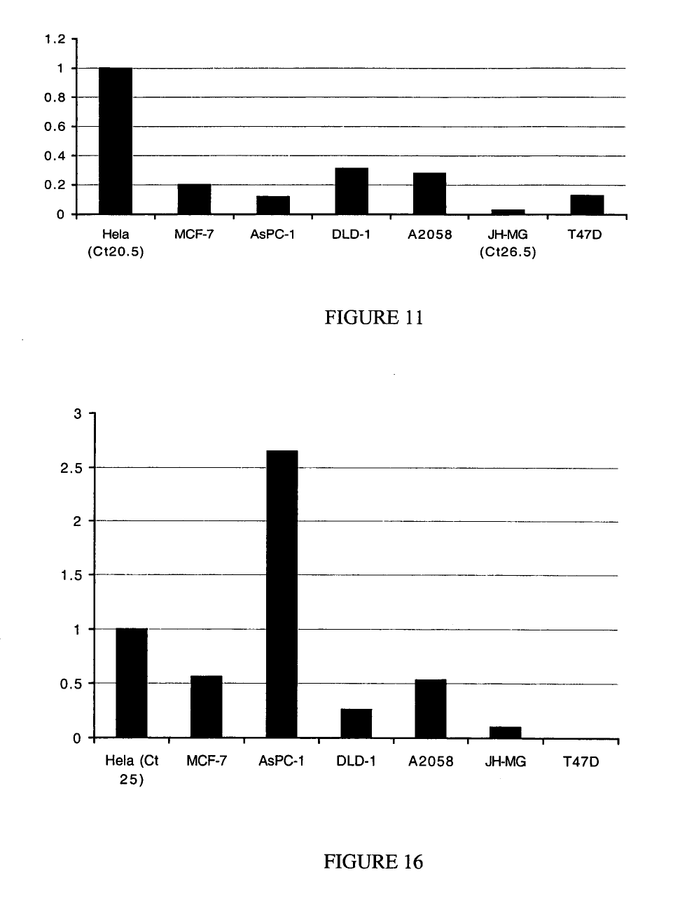

The expression level of NR4A1 mRNA was assessed in the following cell lines: HeLa (cervical cancer), DLD1 (colon cancer), A2058 (melanoma), MCF7 (breast cancer), T47D (breast cancer), and JHMG (glioma). RNA samples from sub-confluent cultures were prepared by standard procedures and real-time PCR (RT-PCR) was performed on these samples. The results are shown in FIG. 11. NR4A1 mRNA level was normalized to the internal ribosomal 18S RNA control level. Relative mRNA levels are depicted as arbitrary units. These results show that NR4A1 is most highly expressed in HeLa cells, but also expressed to a significant degree in all the other cancer cell lines tested.

3. Knockdown of NR4A1 in HeLa Cells by Transfection with siRNA

Three siRNA constructs against NR4A1 (SEQ ID NO:1) and a control siRNA were transiently transfected into HeLa cells using Oligofectamine™. The sense strand of the first siRNA (A) was GUUCGAGGACUUCCAGGUG (SEQ ID NO:15), and the antisense strand was CACCUGGAAGUCCUCGAAC (SEQ ID NO:16). The sense strand of the second siRNA (B) was GGAAGUUGUCCGAACAGAC (SEQ ID NO:17), and the antisense strand was GUCUGUUCGGACAACUUCC (SEQ ID NO:18). The sense strand of the third siRNA (C) was GAUCUUCAUGGACACGCUG (SEQ ID NO:19), and the antisense strand was CAGCGUGUCCAUGAAGAUC (SEQ ID NO:20).

4. Validation Assays

Twenty-four hours after transfection, the cells were trypsinized and plated into 96-well plates. A number of cell-based validation assays were then performed to assess the phenotype resulting from the introduction of these siRNAs into the various cell lines.

a. Anchorage-Dependent Growth in HeLa Cells:

Cell growth in liquid culture was quantified on day 3 by alamarBlue™ staining (BioSource International, Inc., Camarillo, Calif.). The results of two independent experiments with six data points are summarized in FIG. 12A. These results show that each of the three siRNAs targeting NR4A1 caused a significant reduction in anchorage-dependent growth (P-values for all three siRNAs <0.01).

b. Anchorage-Independent Growth in HeLa Cells:

Following one week of soft agar culture, the cells were stained with alamarBlue™. The results of two independent experiments with six data points are summarized in FIG. 12B. These results show that each of the three siRNAs targeting NR4A1 also caused a significant reduction in anchorage-independent growth (P-values for all three siRNAs <0.01).

c. Down-Regulation of NR4A1 mRNA

48 hours after transfection with the three siRNAs targeting NR4A1 and a control siRNA, the cells were harvested and mRNA extracted and subject to real-time RT-PCR (Taqman®) analysis. As can be seen from FIG. 12C, all three siRNAs caused reduction in the NR4A1 mRNA levels which was consistent with the biological phenotype observed in the previous two experiments (reduction in anchorage-independent growth in soft-agar and reduction in anchorage-dependent growth in liquid culture).

5. Growth/Survival Assays in Other Cell Lines.

The effect of NR4A1 knockdown on cancer cell growth or survival was also tested in other cancer cell lines using the same three siRNAs targeting NR4A1 as described above in connection with HeLa cells. FIG. 13A shows that knock-down of NR4A1 by the siRNAs in DLD1 cells caused a significant reduction in anchorage-independent growth, as measured by alamarBlue™ in soft agar. Similar results were obtained for AsPC1 cells (FIG. 13B), U87 cells (FIG. 13C) and A2058 cells (FIG. 13D). FIG. 13E shows that a similar result was obtained when the siRNAs were transiently transfected into PC3 cells, and cell viability/survival was assayed by WST-1 (Roche Molecular Biochemicals, Mannheim, Germany) staining 24 hrs after transfection, with the cells switched to media containing 1% serum and incubated for 4 days.

6. Apoptosis Assays

The reduction in cancer cell growth by the knockdown of NR4A1 evidenced by the assays described above and shown in FIG. 12 and FIG. 13 could result either from an increase in cell death (apoptosis) or a reduction in cell proliferation. To ascertain which of these two processes predominated, two apoptosis-specific assays were conducted—a Caspase 3/7 activity assay that measures the level of activity of the caspase enzyme (an increase in activity indicates an increase in apoptosis), and an ELISA assay that measures the amount of DNA fragmentation of cells that have undergone apoptosis.

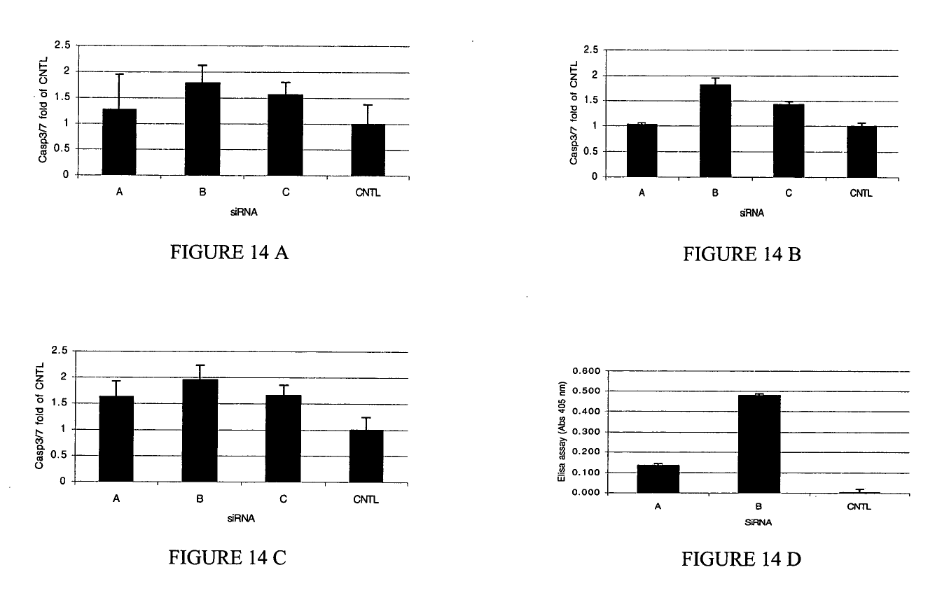

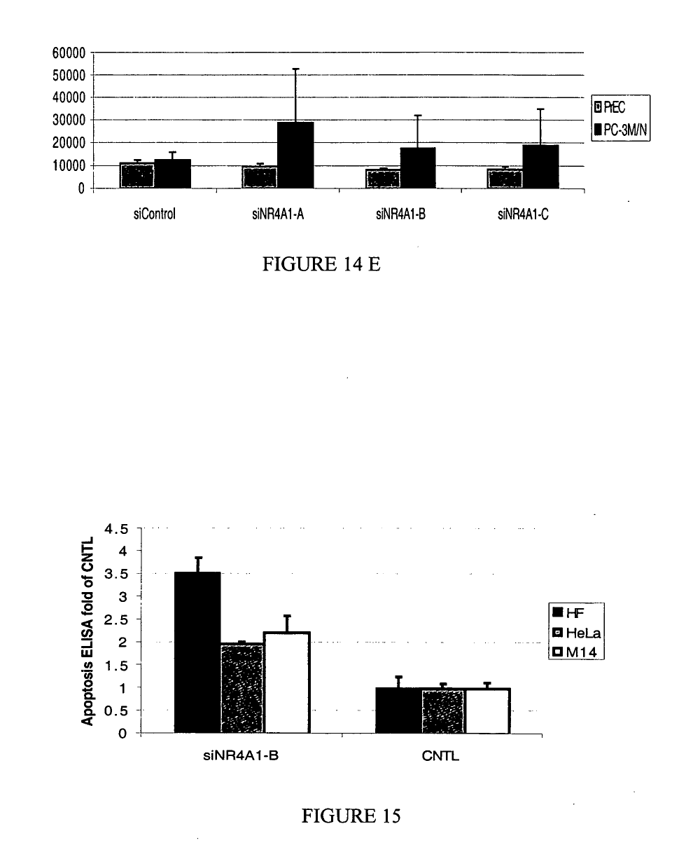

HeLa, DLD1, and U87 cells were seeded in 96-well plates and transfected with 10 nM in vitro transcribed siRNAs on the following day using Oligofectamine™. 48 hours post-transfection, Caspase 3/7 activity was measured using an Apo-ONE kit in accordance with the manufacturer's (Promega) instructions. Assay incubation time was 16 hours. The results of these measurements are shown in FIG. 14A (HeLa), FIG. 14B (DLD1), and FIG. 14C (U87). As can be seen from these figures, knockdown by siRNA of NR4A1 mRNA resulted in a significant increase in Caspase 3/7 activity as compared to a control siRNA.

The results of the Caspase 3/7 assays were confirmed by an ELISA assay on HeLa cells. Two siRNAs (A and B) targeting NR4A1 and a control siRNA were transfected individually into HeLa cells seeded in 12 well plates at approximately 30% confluency, using Oligofectamine™, at a concentration of 10 nM. 24 hours post-transfection, the cells were harvested and assayed by ELISA. As can be seen from FIG. 14D, knockdown of NR4A1 by both siRNAs resulted in a significant increase in cancer cell death (apoptosis) relative to the control.

7. Effect of NR4A1 Knockdown is Cancer-Cell Specific

Moreover, knockdown of NR4A1 results in an increase in cell death (apoptosis) selectively in cancer cells compared to normal (non-cancerous) cells. Specifically, a prostate cancer cell line (PC-3M/N) and a non-cancerous prostate cell line (PrEC) were used for siRNA transfection and Caspase 3/7 analysis as described above. Cells from both cell lines were seeded in 96 well plates. 24 hours later, the cells were transfected with the three siRNAs targeting NR4A1. 3 days post-transfection, the cells were analyzed for caspase activity using the Promega Apo-ONE Homogeneous Caspase 3/7 assay. Activity was read 6 hours post-lysis. The results are shown in FIG. 14E. As can be seen, knockdown of NR4A1 caused apoptotic cell death only in the cancer cells, and did not have an effect on normal cells. This indicates that agents targeting NR4A1 may be preferred candidates for cancer treatment and cancer therapeutics because it can selectively target cancer cells.

8. Knockdown of NR4A1 by Transduction with siRNA

The results of the assays described above, based upon transient transfection with siRNAs targeting NR4A1, were further confirmed by an additional experiment in which HeLa, HF and M14 (melanoma) cells were transiently transduced using a lentiviral construct expressing siRNA B (see paragraph 3 above) as a hairpin siRNA (Ke, N. et al., “One week 96-well soft agar growth for cancer target validation”, BioTechniques 36: 826-833 (2004)). Four days post transduction, cell lysates were made and DNA fragmentation was measured using the Cell Death Detection ELISA Plus kit (Roche Biochemicals). The results (mean from three independent experiments) are shown in FIG. 15.

### EXAMPLE 2

This example shows that knockdown of the nuclear receptor NR4A2 (SEQ ID NO:2; accession #: NM_{—}006186.1) inhibits growth and/or proliferation of cancer cells.

1. General.

NR4A2, also named RNR-1, TONOR, HZF-3, and Nurr1, is a transcription factor belongs to the superfamily of nuclear receptors (NRs). Law, et al., Mol. Endocrinol., 6:2129-2135 (1992). It is a member of the Nur77/NGFIB subfamily of orphan nuclear hormone receptors, that also includes NR4A1 and NR4A3; all three belong to the steroid-thyroid hormone-retinoid receptor subfamily.

Like other nuclear receptors, the structure of NR4A2 includes an activation domain, a DNA binding domain and a ligand binding domain. While its DNA binding domain is highly homologous to that of the other members of the same subfamily (NR4A1 and NR4A3), the ligand-binding domain is modestly homologous, and the activation domain is quite divergent.

NR4A2 and other members of the subfamily bind as a monomer to AAAGGTCA (SEQ ID NO:21) termed NBRE (NGFI-B response element) to activate target gene expression (Cheng 1997; Paulsen 1995; Wilson 1991); NR4A2 also binds AAAT(G/A)(C/T)CA (SEQ ID NO:22) termed NurRE (Nur-responsive element) as dimers and activate transcription of the POMC gene. Maira, et al., Mol. Cell. Biol., 19:7549-7557 (1999). NR4A1 and NR4A2 can also heterodimerize with RXR (retinoic x receptor) and activate transcription through a DR-5 element in a 9-cis retinoic acid-dependent manner. Zetterstrom, et al., Mol. Endocrinol., 10:1656-1666 (1996).

NR4A2 is expressed in the developing and adult central nervous system. It is believed to be involved in the regulation of corticotropin-releasing hormone, cytokine related functions, etc. NR4A2 transcription is activated by NF-kB and cAMP response element in rheumatoid arthritis synovial tissues, and, therefore, implicated in rheumatoid arthritis. McEvoy, et al., J. Immunol., 168:2979 (2002). It is essential for the development of the midbrain dopaminergic neurons as genetic inactivation results in impaired development and maintenance of midbrain dopaminergic neurons (Saucedo-Cardenas, et al., Proc. Natl. Acad. Sci. USA, 95:4013-4018 (1998)). It has also been implicated in cell cycle and morphological differentiation in some cells. The potential targets of NR4A2 include cocaine-sensitive dopamine transproter (DAT) and tyrosine hydroxylase (Giros, et al., Nature, 379:606-612 (1996)).

2. Expression of NR4A2 in Cancer Cells

The expression level of NR4A2 mRNA was assessed in the following cell lines: HeLa (cervical cancer), MCF7 (breast cancer), AsPC1 (pancreatic cancer), DLD1 (colon cancer), A2058 (melanoma), JHMG (glioma), and T47D (breast cancer). See FIG. 16.

3. siRNA Knockdown of NR4A2 in HeLa Cells

Three siRNA constructs against NR4A2 and a control siRNA were transiently transfected into HeLa cells using Oligofectamine™. The sense strand of the first (A) siRNA is CUACAGCACAGGCUACGAC (SEQ ID NO:23) and the antisense strand is GUCGUAGCCUGUGCUGUAG (SEQ ID NO:24). The sense strand of the second (B) siRNA is UCGCUGUCAGUACUGCCGA (SEQ ID NO:25) and the antisense strand is UCGGCAGUACUGACAGCGA (SEQ ID NO:26). The sense strand of the third siRNA (C) is GAGAGUGGAAGAACUGCAA (SEQ ID NO:27), and the antisense strand was UUGCAGUUCUUCCACUCUC (SEQ ID NO:28).

4. Validation Assays

After transfection with siRNA, cell-based validation assays in a 96-well format were performed to assess the phenotype resulting from the introduction into HeLa cells of siRNA targeted to NR4A2.

a. Growth Assays in Hela:

The effect of the knockdown of NR4A2 by siRNAs on the proliferation/growth of HeLa cells was assayed, both in liquid culture (anchorage-dependent growth) and in soft agar (anchorage-independent growth), using procedures substantially similar to those described above in Example 1 in connection with NR4A1. Two of the three siRNAs (B, C) caused a statistically significant reduction in liquid culture growth (anchorage-dependent) (FIG. 17A). The same two siRNAs (B, C) also caused an even larger reduction in soft-agar growth (anchorage-independent growth) (FIG. 17B). Real-time PCR (Taqman®) analysis of HeLa NR4A2 mRNA after transfection with the siRNAs showed a significant reduction in the NR4A2 mRNA levels (FIG. 17C), a result consistent with the biological phenotype observed in the growth assays.

b. Growth/Survival Assay in Other Cell Lines.

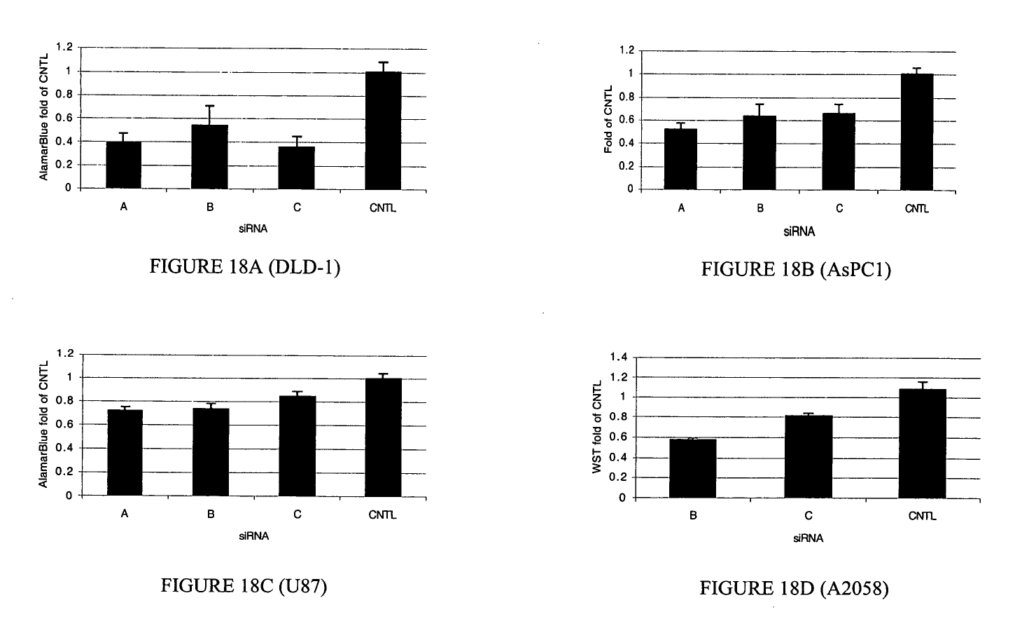

The effect of NR4A2 knockdown on cell growth or survival was also tested in other cell lines. Anchorage independent (soft agar) growth was tested in DLD-1 (FIG. 18A), AsPC1 (FIG. 18B), and U87 (FIG. 18C). Survival in low serum media (1%) was tested in A2058 (FIG. 18D). Cells transfected with the three NR4A2 siRNAs and a control were either plated into 96-well soft agar culture or media containing low serum. Cell growth/survival was scored either with alamarBlue™ staining after one week growth in soft agar, or with WST-1 staining after four days in 1% serum media. All siRNA tested caused either slower growth in soft agar or less viability in low serum media.

5. Transduction Assays

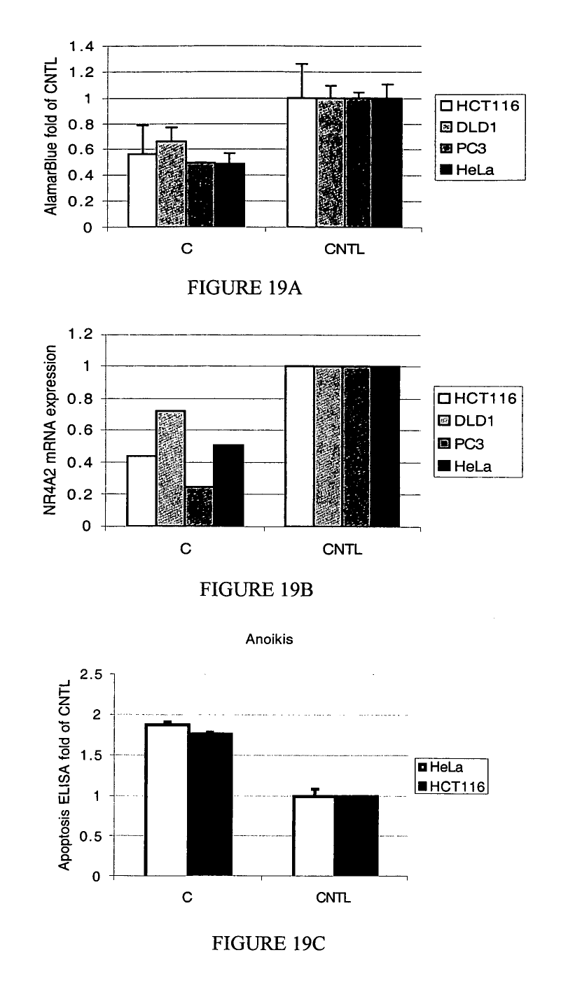

The results described above which were based upon transient transfection with siRNAs were confirmed by a series of experiments in which HeLa, PC3 (prostate cancer), HCT116 (colon cancer) and DLD1 cell lines were stably transduced either with siRNA C (paragraph 3 above) or a control siRNA, using a lentiviral construct as described in Ke et al (Ibid.). After transduction and selection in media containing desired concentrations of puromycin, 1000 cells per well of each type were seeded into soft agar and allowed to grow for one week before being scored by alamarBlue™ staining to measure anchorage-independent growth. Each experiment was performed in triplicate, with the results shown in FIG. 19A (error bars indicate standard deviation). In addition, total RNA was prepared from the stable cells transduced either with the siRNA against NR4A2 or the control siRNA, and the RNA was subjected to the Realtime RT-PCR (Taqman®) analysis to detect NR4A2 mRNA levels. The results of three experiments for each sample are shown in FIG. 19B.

The role of NR4A2 in anoikis (a form of cell death) was also tested using HeLa and HCT116 cells which had been stably transduced with the siRNA against NR4A2. Stable cells were detached from the plates and resuspended in methylcellulose for 16 hours. Cell lysates were then made and DNA fragmentation was measured using the Roche Biochemicals Cell Death Detection ELISA Plus kit. The results (mean values from three independent experiments) are shown in FIG. 19C.

### EXAMPLE 3

This example shows that knockdown of the nuclear receptor NR4A3 (SEQ ID NO:3; accession #: NM_{—}006981) inhibits growth or proliferation of cancer cells.

1. General

NR4A3 (Nuclear Receptor Subfamily 4, Group A, Member 3), or Neuron derived orphan receptor-1 (NOR-1), MINOR (mitogen inducible nuclear orphan receptor), TEC, is an orphan nuclear hormone receptor of unknown function. It belongs to the steroid-thyroid hormone-retinoid receptor superfamily, and can efficiently bind the NGFI-B Response Element (NBRE). The encoded protein may act as a transcriptional activator. It can be induced by mitogen in certain cells, similar to NGFI-B/Nur77, another mitogen-inducible orphan receptor. While NR4A3 gene expression is inhibited in Jurkat cells by the immunosuppressant cyclosporin A, NGFI-B/Nur77 is not, suggesting that they are regulated by distinct pathways. NR4A3 and other orphan receptors bind to the same sequence of AAAGGTCA (SEQ ID NO:21), NBRE (NGFI-B response element), and share a conserved DNA-binding domain. However, there is marked divergence in other domains, particularly the N-terminal putative transactivation domain. When NR4A3 is co-expressed with submaximal levels of NGFI-B/Nur77, synergistic or additive levels of target gene expression are obtained in a target reporter assay. However, at maximal levels of NGFI-B/Nur77 expression, NR4A3 antagonizes the reporter gene expression in a dose-dependent fashion.

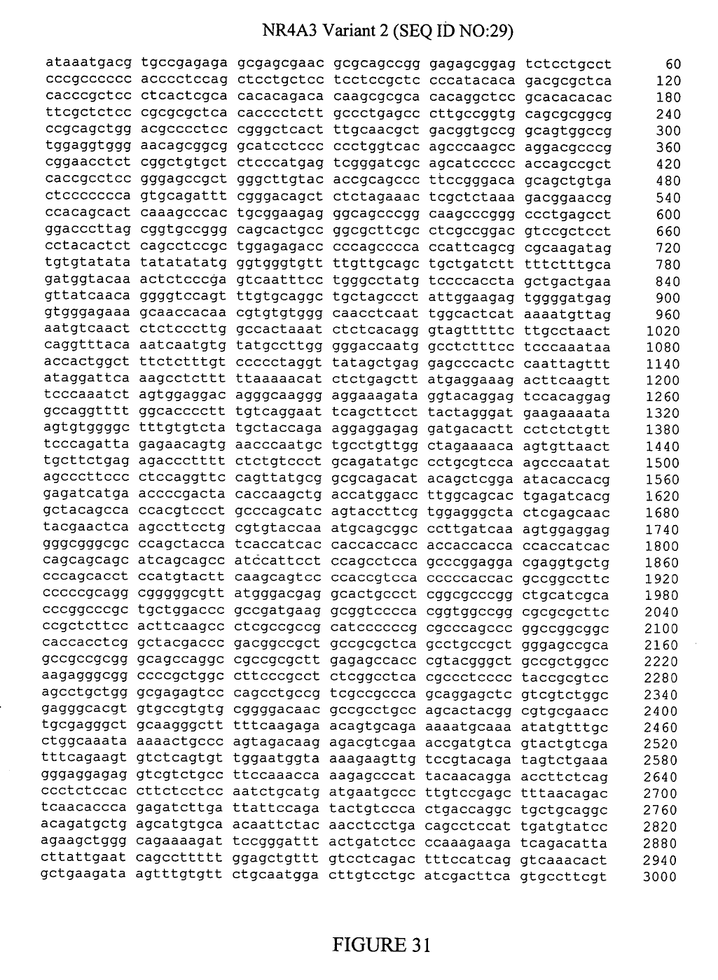

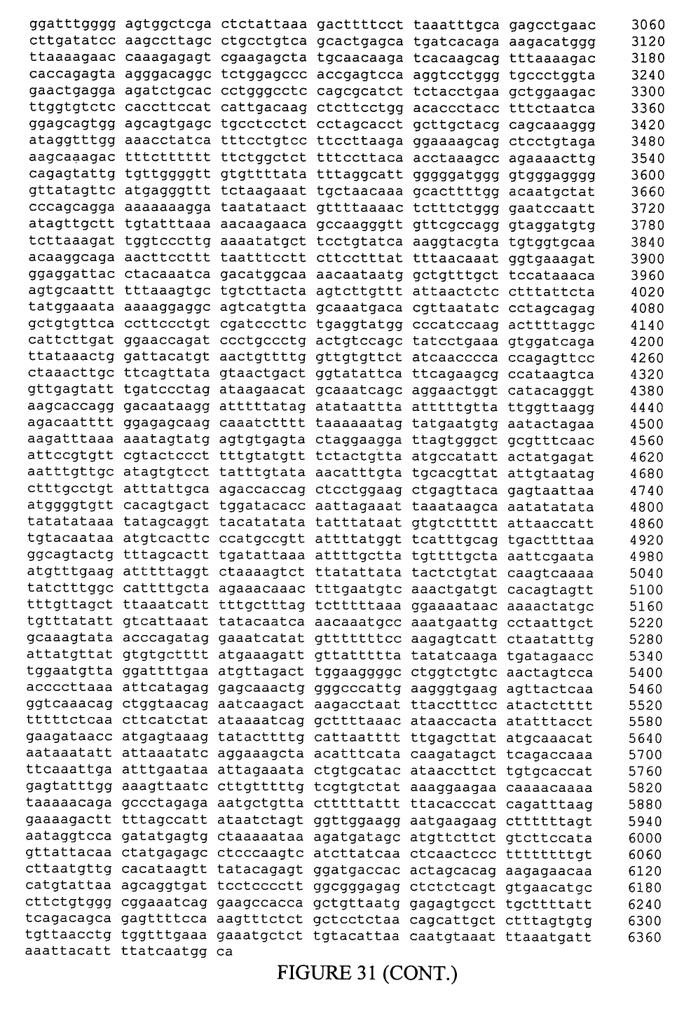

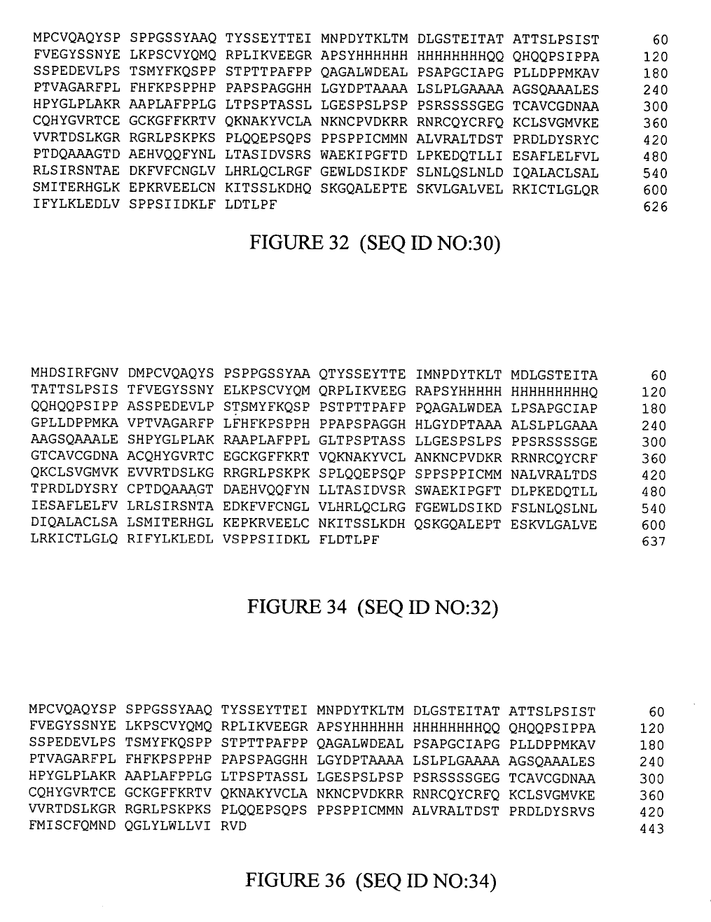

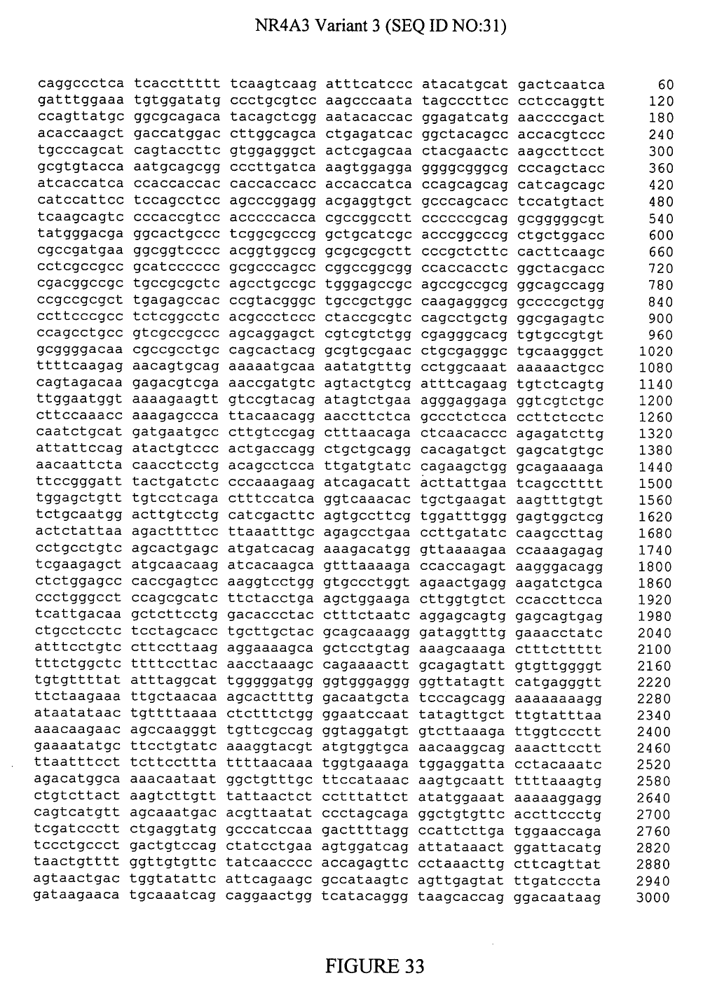

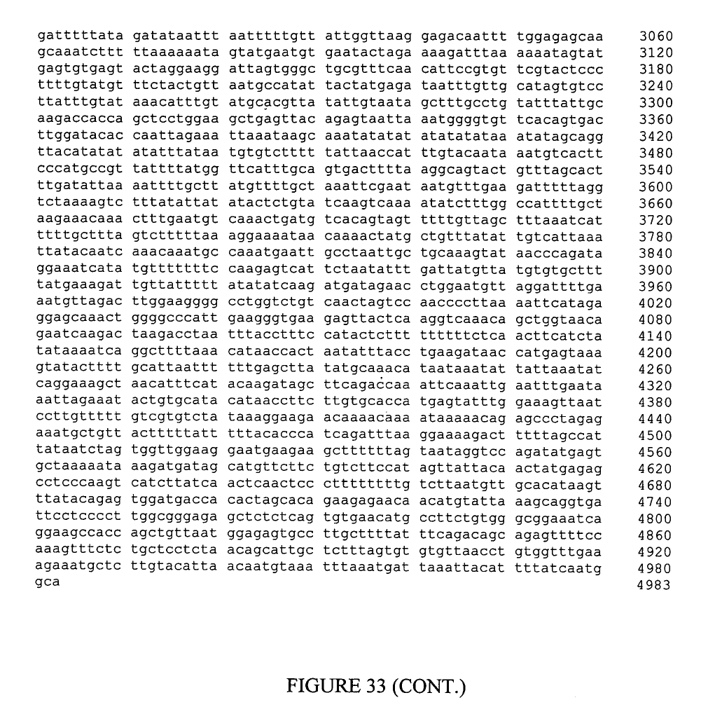

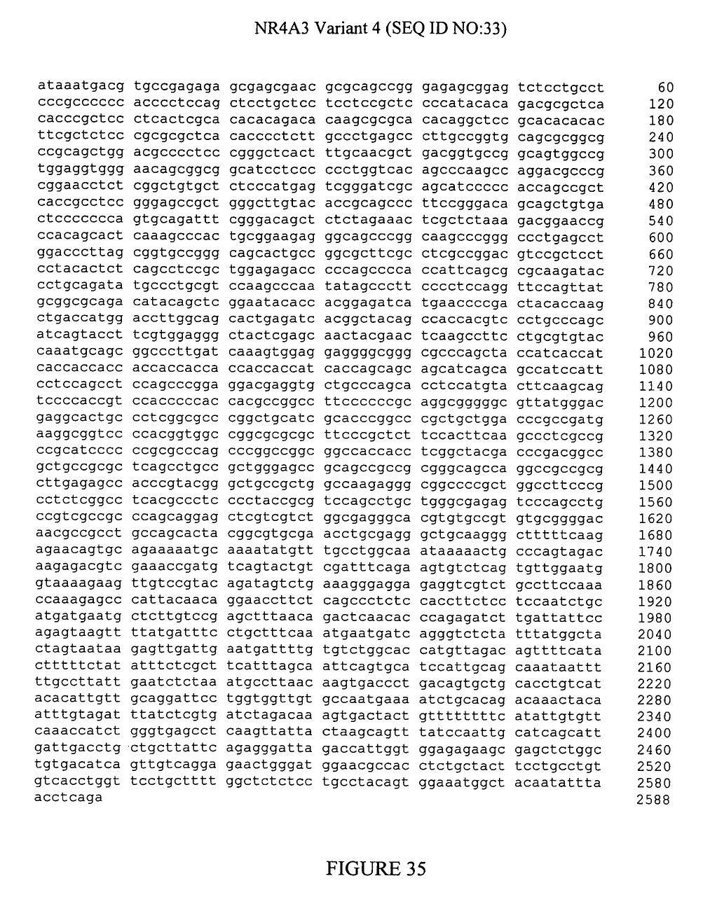

NR4A3 mRNA has been detected in the adult heart and skeletal muscle as well as in the fetal brain, indicating that its expression is not restricted to events that occur during neural development. The gene is 35 kilobases long, interrupted by seven introns. The exon-intron structure of the gene is generally conserved when compared with the steroid/thyroid receptor superfamily and is remarkably similar to that of the Nur77/NGFI-B genes. FISH revealed that the gene is located on chromosome 9q. Four transcript variants (SEQ ID NO:3; SEQ ID NO:29 (see FIG. 31); SEQ ID NO:31 (see FIG. 33); and SEQ ID NO:33; see FIG. 35) encoding three distinct isoforms have been identified for this gene. Isoform b (SEQ ID NO:31; NM_{—}173200) differs in the 5′ UTR and coding region compared to isoform a (SEQ ID NO:3; NM_{—}006981), which results in the encoded protein isoform b (SEQ ID NO:30; see FIG. 32) containing a longer N-terminus compared to isoform a. Variant 4 encodes isoform c (SEQ ID NO:34; see FIG. 36). The present invention encompasses all variants, RNA correlates thereof and all isoforms with respect to all methods described herein.

2. Expression of NR4A3 in Cancer Cells

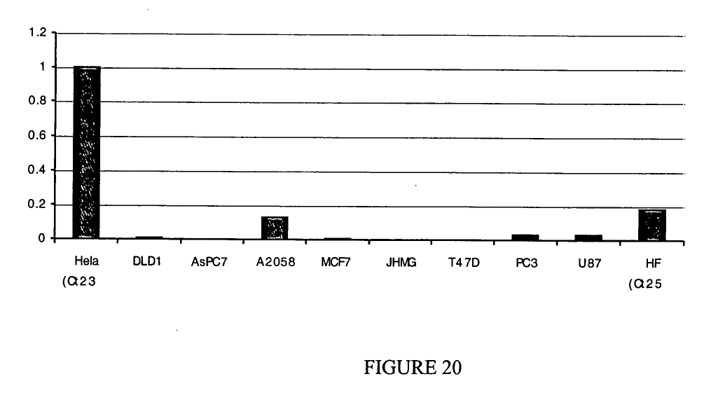

The expression level of NR4A3 mRNA was assessed in various cancer cell lines. As can be seen from FIG. 20, the most significant expression of NR4A3 mRNA was in HeLa cells.

3. siRNA Knockdown of NR4A3 in HeLa Cells

Expression vectors encoding several different siRNAs against NR4A3 and a control siRNA were generated. HeLa cells were then transiently transfected with each of these vectors, and the siRNAs were expressed in the cells. The sense strands of three of these siRNAs were as follows:

| | | |
|---|---|---|
| (D): | GUGUUCUGCAAUGGACUUGUC; | (SEQ ID NO: 35) |
| (E): | GACAAGCTCUUCCUGGACACC; | (SEQ ID NO: 36) |
| and | | |
| (F): | GUUCACCUUCCCUGUCGAUCC. | (SEQ ID NO: 37) |

4. Validation Assays

The effect of the knockdown of NR4A3 by siRNAs on the proliferation/growth of HeLa cells was assayed, both in liquid culture (anchorage-dependent growth) and in soft agar (anchorage-independent growth), using procedures substantially similar to those described above in Example 1 and Example 2.

No reduction in anchorage dependent (liquid culture) growth was observed. However, both siRNA (D) and siRNA (E) caused substantial reduction in anchorage-independent (soft agar) growth, as can be seen from FIG. 21A. This biological phenotype change was correlated with the down-regulation of NR4A3 mRNA, as evidenced by Taqman® analysis (FIG. 21B).

### EXAMPLE 4

This example shows that knockdown of the nuclear receptor NR2F6 (SEQ ID NO:4; Accession #: NM_{—}005234) inhibits growth/proliferation of cancer cells.

1. General

NR2F6 (also known as Ear2 or ERBAL2) is an orphan nuclear hormone receptor. It was originally identified (Miyajima et al., Nucleic Acids Res., 16:11057-11073 (1988)) as an ErbA related gene, and was found to contain homology to steroid and hormone receptors. The longest sequence for NR2F6 is 2380 bp in length and contains four exons (Accession#X12794). The chromosomal location for this gene is 19p13.1. It is a member of the COUP-TF (NR2F1) subfamily of nuclear receptors, which have been reported to interfere with the signaling pathways of several nuclear receptors including retinoic acid, thyoid hormone, estrogen and vitamin D3 receptors. Members of the COUP-TF subfamily of nuclear receptors are transcription factors and generally considered to be transcription repressors.

2. siRNAs Against NR2F6

Five siRNAs against NR2F6 and a control siRNA were constructed with the following sense strands:

| | | |
|---|---|---|
| (A) | AACUUGCGGCCAAACUUUCCC | (SEQ ID NO: 38) |
| (B) | AACUUUUUGGGGGUGCAGC | (SEQ ID NO: 39) |
| (C) | AAGCAUUACGGUGUCUUCACC | (SEQ ID NO: 40) |
| (D) | AACCGUGACUGCCAGAUCGAC | (SEQ ID NO: 41) |
| (E) | AAGACGCCCAUUGAGACUG | (SEQ ID NO: 42) |

3. NR2F6 Knockdown in A172 (Gliobastoma) Cells

The effect of siRNA knockdown of NR2F6 was first tested in A172 glioblastoma cells. The five NR2F6 siRNAs and a control siRNA were transcribed in vitro. The siRNAs (conc. 15 nM) were transfected a day after seeding cells in 96 well plates. For each individual siRNA, 6 wells were transfected, along with the control scrambled siRNA. Two days after transfection, the cells were treated with 10 μM C₂ ceramide. 18 hours after the addition of C₂ ceramide, the plates were analyzed for cell survival using WST-1. The data (percentage survival based on the WST-1 results) as shown in FIG. 22, indicate that up to a five-fold increase in apoptosis resulted from the knockdown of NR2F6. Taqman® analysis (not shown) of NR2F6 mRNA after transfection with the siRNAs correlated with these findings.

4. NR2F6 Knockdown in JH-MG (Glioblastoma) Cells

The effect of siRNA knockdown of NR2F6 was then evaluated in the JH-MG glioblastoma cell line.

Four NR2F6 siRNA constructs (A, B, C and D) and the control siRNA were transcribed in vitro. JH-MG cells were transfected in duplicate wells of a 12 well plate with 10 nM siRNA one day after seeding. Two days after transfection, the cells were treated with Fas to induce apoptosis. A DNA fragmentation ELISA was used to assess the amount of Fas induced apoptosis in each sample. As a positive control for the assay, cells were treated with a combination of Actinomycin D and Fas for maximal induction of apoptosis. The results are shown in FIG. 23 (data graphed as fold over control to normalize it from different experiments). As can be seen, knockdown of NR2F6 by one of the siRNAs resulted in a greater than ten-fold increase in Fas-induced apoptosis relative to the control. Taqman® analysis of NR2F6 mRNA after transfection with the siRNAs correlated with these findings (not shown).

5. NR2F6 Knockdown in AsPC1 (Pancreatic Cancer) Cells

The effect of siRNA knockdown of NR2F6 was also tested in a pancreatic cancer cell line, AsPC1. Two of the NR2F6 siRNA constructs (A and C; SEQ ID NOS:38 and 40) and control siRNA were transcribed in vitro. AsPC1 cells were transfected in duplicate wells of a 12 well plate with 10 nM siRNA one day after seeding. Two days after transfection, the cells were treated with Fas (to induce apoptosis). A DNA fragmentation ELISA was used to assess the amount of Fas induced apoptosis in each sample. As a positive control for the assay, cells were treated with a combination of Actinomycin D and Fas for maximal induction of apoptosis. The results are shown in FIG. 24 (data graphed as fold over control to normalize it from different experiments). As can be seen, knockdown of NR2F6 by both siRNAs resulted in a significant increase in Fas-induced apoptosis. Taqman® analysis of NR2F6 mRNA after transfection with the siRNAs correlated with these findings.

6. NR2F6 Knockdown in DLD-1 (Colon Carcinoma) Cells

The effect of siRNA knockdown of NR2F6 was also tested in the DLD-1 colon carcinoma cell line. Experiments similar to those described above with respect to the glioblastoma and pancreatic cells lines were conducted, but these did not elicit the same induction of cell death as was observed in the glioblastoma and pancreatic cell lines. This negative data correlates well with epidemiological information indicating that NR2F6 is not up-regulated in colon cancer cells relative to normal (non-cancerous) colon cells.

7. Ribozyme Knockdown of NR2F6

A172 (glioblastoma) cells were transduced with three separate ribozymes engineered to target NR2F6 and two control ribozymes. The sequences of the ribozymes targeting NR2F6 were as follows:

(SEQ ID NO: 43)

CAGGUGAAAGAACCGUACCAGAGAAACACACGUUGUGGUAUAUUAC
CUGGUA;

(SEQ ID NO: 44)

CTTCTTGAAGAAGCAGACCAGAGAAACACACGUUGUGGUAUAUUAC
CUGGUA;
and

(SEQ ID NO: 45)

ATGTGTCTAGAATAGGACCAGAGAAACACACGUUGUGGUAUAUUAC
CUGGUA.

After growing in selection media (containing puromycin) for six days, the cells were treated with or w/o c₂-ceramide, and the colony numbers/cm² were counted the next day. The results showed that knockdown of NR2F6 by each of the ribozymes caused increased sensitization to c₂-ceramide induced apoptosis.

### EXAMPLE 5

This example shows that knockdown of the nuclear receptor NR2F1 (SEQ ID NO:5; Accession #: NM_{—}005654) inhibits growth or proliferation of cancer cells.

1. General

NR2F1, or COUP-TFI (chicken ovalbumin upstream promoter-transcription factor I), is an orphan nuclear hormone receptor of unknown function. The gene encoding NR2F1 is located at chromosomal locus 5q15. It encodes a protein of 423AA with two domains: a ZnFC4 domain (the c4 zinc finger in nuclear hormone receptors (83 to 154AA); and a HOLI domain (ligand binding domain of hormone receptors (221 to 381AA).

2. siRNAs Against NR2F1

Three in vitro transcribed siRNAs against NR2F1 were generated. Their sense and antisense strands were as follows:

| | | | |
|---|---|---|---|
| A) | Sense | UACUGCCGCCUCAAGAAGU | (SEQ ID NO: 46) |
| | strand: | | |
| | Antisense | ACUUCUUGAGGCGGCAGUA | (SEQ ID NO: 47) |
| | strand: | | |
| B) | Sense | GGCGCUACACGUCGACUCA | (SEQ ID NO: 48) |
| | strand: | | |
| | Antisense | UGAGUCGACGUGUAGCGCC | (SEQ ID NO: 49) |
| | strand: | | |
| C) | Sense | CUGGCCUUACAUGUCCAUC | (SEQ ID NO: 50) |
| | strand: | | |
| | Antisense | GAUGGACAUGUAAGGCCAG | (SEQ ID NO: 51) |
| | strand: | | |

3. NR2F1 Knockdown in A2058 (Melanoma) Cells

The effect of siRNA knockdown of NR2F1 in A2058 (melanoma) cells was tested. A2058 cells were seeded in 96-well plates and transfected with 10 nM in vitro transcribed siRNAs on the following day, using 0.4 μl oligofectamine. 24 hours post-transfection, media was changed to 1% serum (growth factor deprivation). 48 hours after transfection, cells were treated either with 1% serum or 1% serum/Brefeldin A (an apoptosis inducer) for 3 days. Cell viability was measured using the WST-1 reagent. The results show that in 1% serum (FIG. 25A), knockdown of NR2F1 by one of the three siRNAs (C) caused a significant increase in sensitization to apoptosis; and in the combination of 1% serum/Brefeldin A (FIG. 25B), knockdown of NR2F1 by all three siRNAs caused a significant increase in sensitization to apoptosis.

4. NR2F1 Knockdown in HeLa, DLD-1, and U87 Cells

The effect of knockdown of NR2F1 by the same three siRNAs was also tested in three additional cell lines (HeLa, DLD-1 and U87). The siRNA were transfected into cells in 96-well plate format using Oligofectamine™. The cells were then harvested and an equivalent number of cells was seeded in 96 well plates for liquid and soft-agar cultures. Following a week of soft-agar culture, the cells were stained with alamarBlue™. The experiments were repeated twice and six data points were analyzed. The results are shown in FIG. 26A (HeLa), FIG. 26B (DLD-1), and FIG. 26C (U87). These results demonstrate that knockdown of NR2F1 by siRNA reduces both anchorage independent and anchorage dependent growth in these cell lines.

All references made herein, including articles, patent applications and any other publications, are incorporated by reference in their entirety.

1. A method of identifying a molecule that inhibits cancer cells, comprising:
a. introducing said molecule into cells, wherein said molecule binds to a compound comprising SEQ ID NOS: 2, 1, 3, 4 or 5 or comprising the RNA correlate of SEQ ID NOS: 2, 1, 3, 4 or 5; and
b. measuring the level of inhibition of said cells, where an increase in level indicates said molecule inhibits cancer cells.

2. The method of claim 1, wherein said compound comprises SEQ ID NOS: 2, 1, 3, 4 or 5.

3. The method of claim 1, wherein said compound comprises the RNA correlate of SEQ ID NOS: 2, 1, 3, 4 or 5.

4. The method of claim 1, wherein said compound consists of SEQ ID NOS: 2, 1, 3, 4 or 5.

5. The method of claim 1, wherein said compound consists of the RNA correlate of SEQ ID NOS: 2, 1, 3, 4 or 5.

6. The method of claim 1, wherein said inhibition is measured by an apoptosis assay, where an increase in the level of apoptosis indicates that said molecule inhibits said cells.

7. The method of claim 1, wherein said inhibition is measured by a proliferation assay, where a decrease in the rate of cell division indicates that said molecule inhibits said cells.

8-9. (canceled)

10. A method of identifying a molecule that inhibits cancer cells, comprising:
a. introducing said molecule into cells, wherein said molecule binds to a compound comprising an amino acid sequence selected from the group consisting of amino acid 260 to amino acid 331 of SEQ ID NO:7, amino acid 408 to amino acid 566 of SEQ ID NO:7, amino acid 264 to amino acid 335 of SEQ ID NO:6, amino acid 408 to amino acid 566 of SEQ ID NO:6, amino acid 289 to amino acid 360 of SEQ ID NO:8, amino acid 439 to amino acid 594 of SEQ ID NO:8, amino acid 53 to amino acid 124 of SEQ ID NO:9, amino acid 204 to amino acid 364 of SEQ ID NO:9, amino acid 83 to amino acid 154 of SEQ ID NO:10 and amino acid 221 to amino acid 381 of SEQ ID NO:10; and
b. measuring the level of inhibition of said cells, where an increase in level indicates said molecule inhibits cancer cells.

11. The method of claim 10, wherein said molecule binds to a compound selected from the group consisting of amino acid 260 to amino acid 331 of SEQ ID NO:7, amino acid 408 to amino acid 566 of SEQ ID NO:7, amino acid 264 to amino acid 335 of SEQ ID NO:6, amino acid 408 to amino acid 566 of SEQ ID NO:6, amino acid 289 to amino acid 360 of SEQ ID NO:8, amino acid 439 to amino acid 594 of SEQ ID NO:8, amino acid 53 to amino acid 124 of SEQ ID NO:9, amino acid 204 to amino acid 364 of SEQ ID NO:9, amino acid 83 to amino acid 154 of SEQ ID NO:10 and amino acid 221 to amino acid 381 of SEQ ID NO:10.

12. The method of claim 10, wherein said molecule binds to a compound comprising a sequence selected from the group consisting of SEQ ID NOS: 7, 6, 8, 9 and 10.

13. The method of claim 10, wherein said molecule binds to a compound selected from the group consisting of SEQ ID NOS: 7, 6, 8, 9 and 10.

14. The method of claim 10 wherein said inhibition is measured by an apoptosis assay, where an increase in the level of apoptosis indicates that said molecule inhibits said cells.

15. The method of claim 10, wherein said inhibition is measured by a proliferation assay, where a decrease in the rate of cell division indicates that said molecule inhibits said cells.

16. A method of inhibiting cancer cells, comprising introducing into said cells a molecule that binds to a compound comprising SEQ ID NOS: 2, 1, 3, 4 or 5, or comprising the RNA correlate of SEQ ID NOS: 2, 1, 3, 4 or 5, whereby the level of cell inhibition is increased.

17. The method of claim 16, wherein said compound comprises SEQ ID NOS: 2, 1, 3, 4 or 5.

18. The method of claim 16, wherein said compound comprises the RNA correlate of SEQ ID NOS: 2, 1, 3, 4 or 5.

19. The method of claim 16, wherein said molecule is siRNA.

20-26. (canceled)
